(19) United States
(12) Patent Application Publication
(10) Pub. No.: US 2009/0171448 A1
(43) Pub. Date: Jul. 2, 2009

(54) Implantable device with miniature rotating portion for energy harvesting

(76) Inventor: Uri Eli, Kiryat Ono (IL)

Correspondence Address: Dr. D. Graeser Ltd.;c/o The Discovery Dispatch, 9003 Florin Way, Upper Marlboro, MD, 20772, US

(21) Appl. No.: 11/976,434

(22) Filed: Oct. 24, 2007

Related U.S. Application Data
(60) Provisional application No. 60/924,041, filed on Apr. 27, 2007.

(51) Int. Cl. A61F 2/06; A61N 1/00

(52) U.S. Cl. 623/1.32; 607/116

(57) ABSTRACT

A miniature rotating portion, anchored to and used within the human body. Optionally and preferably, the device may be used to generate energy for sensors or other implantable devices.

This Application claims priority from U.S. Provisional Application No. 60/924,041, filed on Apr. 27, 2007, which is hereby incorporated by reference as if fully set forth herein.

### FIELD OF THE INVENTION

The present invention relates to implantable devices with at least one miniature rotating portion and optionally at least one filtering portion and the various uses and methods thereof, and in particular to such devices for use in the human body for energy harvesting.

### BACKGROUND OF THE INVENTION

There are a growing number of implanted medical devices which require miniaturized long lasting power sources. A variety of different types of power sources and batteries have been developed for these implantable devices. Although these power sources provide power for extended periods of time, they periodically still require replacement which involves further surgery on the subject.

Harvesting energy from the freely available energy inherent in the human body can be of great value for powering pacemakers for example. Energy harvesting devices can effectively provide “self powering medical devices” that are far more robust and longer lived than ones powered by a storage battery and/or can support hybrid systems with reduced size batteries. These energy harvesting devices can harvest energy in environments that are difficult or dangerous to access such as the cardiovascular system. This enables them to operate in environments where regular maintenance, to change batteries for example, might be impractical or impossible.

Given the rapid advances in the field of low power consumption of implanted medical devices over the last few years, energy harvesting technology is expected to find wide application in the near future.

Nanotechnology for medical use may be broadly defined as a research field that strives to create miniaturized nano-scale devices that may operate within the human body. The benefits of developing such nanotech devices may be particularly realized in imagining the possible utility in medical applications such as site or target specific diagnosis and monitoring drug delivery and imagery. One of the driving forces to develop nanotech devices is the need to provide better medical solutions for various diseases. Researchers have developed a broad range of nano-scale devices with various applications, but their use has been limited due to limitation in energy source available to power them. Conventional batteries make the nano-scale systems too large, and the toxic contents of batteries limit their use in the body.

Recently, a nanotechnology based device was reported, which attempted to produce energy from a nontoxic human resource including a fluid, in the Apr. 14, 2006 issue of Science (Wang et al). This device features a technique for powering nanometer-scale devices. The researchers reported their findings about a method for converting the body's mechanical energy into electrical power, having potential applications in powering nanotech devices. Reportedly, this method uses bodily movements, such as voluntary muscle contraction, across an array of zinc-oxide (ZnO) nanowires to create an electric potential. The electrical energy is generated by creating a potential utilizing the piezoelectric and semiconductor properties of the ZnO nanowires. Although this technique is in the nano-scale range, it is in its early stages and has a number of disadvantages; in particular the energy produced by this technology is relatively small and non-continuous.

### SUMMARY OF THE INVENTION

There is an unmet need for, and it would be highly useful to have, a device that is able to harvest energy from the body and provide reliable power to implanted medical devices.

The present invention overcomes these drawbacks of the background art by harnessing a small portion of the freely available hydrokinetic energy inherent in the human body fluid flow, such as blood flow for example, and converting it into mechanical, electrical, electrostatic, optical, magnetic, or RF power or other forms of useable and preferable transmittable energy.

The different optional embodiments of the present invention provide for a device and method for harvesting energy from fluid flow inherent and freely available in the human body and supplying the generated energy to implanted medical devices.

Harvesting energy from the freely available hydrokinetic energy inherent in the human body, for example from blood flow, may optionally and preferably be used for any of powering pacemakers, implanted cardioverter defibrillators (ICD) and remote sensors, as well as for optionally transmitting telemetry data through wireless transmitters, neurostimulators, and many other uses.

According to preferred embodiments, such a device could also optionally provide a communication gateway to and from miniature and nano-scale medical devices, providing continuous monitoring and sensing of vessel blood flow rate.

According to preferred embodiments, there is provided a device comprising a vessel support structure, at least one rotating portion, and an electronic portion, wherein the at least one rotating portion and the electronic portion are supported by the vessel support structure. Optionally, these components may be integrated within one another, producing a single unit comprising all three components. Optionally and alternatively each of the components may be differentially attached or coupled to one another forming any number of combinations therefrom. For example one such conformation may comprise a vessel support structure coupled to a rotating portion having an electronic portion incorporated internally. Optionally a vessel support structure may encase at least a part of a electronic portion.

Optionally the rotating portion and electronic portion may be integrally formed within the vessel support structure. Preferably the rotating portion and electronic portion is attached or coupled thereto either during manufacture, or after implanting the vessel supporting structure within a blood vessel or any other bodily passageway.

The vessel support structure and the rotating portion of the device are optionally and preferably made of a dilatable and/or otherwise self-expanding structure that is optionally shaped according to the vessel, including but not limited to a tubular structure, stent like structure, off the shelf (OTS) stent, Stent-Graft or filter structure, or wire frame or other like vessel support structure. Various optional and preferable embodiments, use variation and implementations of this basic structure are presented herewith.

The present invention overcomes the disadvantages of the background art by, among its many advantages, providing in some embodiments a device comprising at least one miniature rotating portion, optionally anchored to a vessel support structure used mainly within the cardiovascular system. Optionally the device of the present invention may be implanted within the cardiovascular system, for example, in vessels such as the aorta, arteries, veins or any blood vessel or other bodily passageways having a flowing fluid.

Any of the embodiments of the present invention may be deployed in a blood vessel. The deployment site may be optionally and preferably determined by any number of imaging methods known in the art and incorporated herein by reference for example including but not limited to X-ray fluoroscopy, intravascular ultrasound, echocardiography, MRI (magnetic resonance imaging), angioscopy, CT (computerized tomography) scan, and/or any other suitable imaging technology. Another optional mode of deployment is surgical, preferably by direct insertion of the catheter carrying the device through a puncture of the targeted vessel in proximity to the deployment site.

Vessel support structures such as stents are implanted within blood vessels and are therefore able to accommodate blood flow and circulation. Blood circulates in the human body due to a pressure gradient that drives the fluid from an area of high pressure to an area of relatively lower pressure. In the human body, blood circulates by the pumping action of the heart in a repetitive predictable manner, from the ventricles returning to the atria, due to the blood pressure gradient that exists between them. When the heart beats, the pressure in the arteries increases to a maximum, thereby creating the systolic pressure gradient that drives blood through the vessels. It is this pressure gradient that enables the blood flow and creates the hydrokinetic energy source that may be harnessed by the present invention.

Similarly, during lower diastolic blood pressure, the rotating portion of a preferred embodiment of the present invention preferably continues to work such that its functional ability is not limited or at least is not blocked by the level of pressure gradient. Furthermore, a preferred embodiment of the present invention is able to continue working for a period of at least a few seconds with zero blood flow, due to the momentum of the rotating portion blades. This enables the rotating portion to work continuously regardless of the blood pressure phase.

The rotating portion, according to a preferred non limiting embodiment of the present invention, is able to harness the hydrokinetic energy of blood flow that causes the rotating portion blades to spin. The rotating portion of the preferred embodiment is preferably stably connected to a vessel support structure for example including but not limited to a stent like structure, stent-graft, filter structure, wire frame or other like vessel support structure. Optionally, the rotating portion is stably connected to the frame of a vessel support structure in a number of optional configurations preferably including but not limited to radial installation within the vessel support structure, wherein the rotation portion is within the lumen of the vessel support structure. Optionally, the rotation portion may be coupled to the frame of the vessel structure wherein the rotating portion is optionally located extra-luminally, outside the lumen of the vessel support structure. Optionally, the rotating portion may be integrated within the plane of the vessel support structure's frame, preferably continuously integrated within the frame of the support structure.

The rotating portion according to a preferred non limiting embodiment of the present invention is optionally and preferably able also to function to clear the vessel support structure and its vicinity of any accumulated plaques and occluding material that may have formed while preferably preventing any new plaques and occluding material from reforming.

The rotating portion according to a preferred non-limiting embodiment of the present invention was developed by applying fluid energy harvesting technology currently only available on a very large macro scale, such as that described in U.S. Pat. No. 1,820,529 to Darrieus, to miniature scale. However, the present invention goes beyond a mere downscaling of the fluid rotating portion described by Darrieus. Adapting the large scale rotating portion to the miniature scale is not simply accomplished as the engineering in making the transition is not a simple extrapolation; furthermore, the biological interaction, blood vessels geometry and mechanics of blood flow further complicates the engineering process. Thus the present invention represents a significant inventive leap over the background art.

A further non limiting preferred embodiment of the present invention is a small scale implantable device that generates power that may optionally be transmitted to other small scale or nano-scale devices, for example according to a mechanism such as wired or wireless coupling, or any other suitable mechanism. Power is generated by continuous, involuntary bodily processes, preferably including but not limited to blood flow.

A non limiting embodiment of the rotating portion of the present invention allows the rotating portion to produce mechanical, electrical, magnetic, optical, RF and/or electrostatic energy or other forms of useable and preferable transmittable energy from a flowing fluid.

The rotating portion according to a preferred non limiting embodiment of the present invention obtains at least a portion of the potential energy available in the hydrokinetic energy of the blood flowing through the device. The power production according to a preferred non limiting embodiment of the present invention may be estimated when considering the available energy in blood flow as calculated in energy per unit time as shown in the equation below, where the potential available power is a function of the area through which the fluid passes and the fluid velocity.

$$P = \frac{1}{2} ρ A {υ}^{3} .$$

P is the potential power, A is the area (m²) through which the blood passes, v is the fluid's velocity (m/sec ) and p is the fluid's density in kg/m³. Applying this equation to the findings in a recent study exemplifies the amount of potential energy available in blood flow.

In a study conducted by the Department of Anesthesia, Indiana University School of Medicine, Richard L. Roudebush VA Medical Center (Indianapolis, Ind., USA), the blood flow velocities in the common carotid and femoral arteries were measured as part of a study measuring the blood flow velocities before and after a direct laryngoscopy and tracheal intubation study. This study was reported in the September 1994 issue of Anesthesia & Analgesia, Vol 78, 1144-1148. They reported that for common carotid artery (CCA) measured at rest in adult men, mean average measured velocity is 49.4 cm/sec (0.494 m/sec); from medical studies, it may be assumed that the mean average of adult men common carotid artery diameter is approximately 7 millimeter (7 mm); p the density of blood is 1060 kg/m³ which therefore estimates the available hydrokinetic power of the common carotid artery is 2.45 milliwatt (2450 μ-watt).

Similarly, in the femoral artery, measurements were taken during rest in adult men, with the mean average measured velocity 107.6 cm/sec (i.e. 1.076 m/sec); from medical studies, it may be assumed that the mean average of adult men femoral artery diameter is approximately 7 millimeter (7 mm); ρ the density of blood is 1060 kg/m³; the calculated available hydrokinetic power of blood flow through the carotid artery is 25.4 milli-watt (25,400 μ-watt).

It should be noted that numerous studies of blood flow velocities have been conducted, and the above data is merely an example of the potential hydrokinetic energy exist in the blood flow. Regardless of the exact calculations, it is clear that hydrokinetic energy will be available to the rotating portion of the present invention. The rotating portion in accordance with the present invention preferably converts the freely available energy inherent in the human body environment into controllable mechanical and electrical power. Harvesting even a small percentage of the above estimated power will allow the rotating portion of a preferred non limiting embodiment of the present invention to reliably provide at least one of electrical, mechanical, magnetic, optical, RF and electrostatic energy or other forms of useable and preferable transmittable energy to at least one medical device associated with it.

The rotating portion according to a preferred non limiting exemplary embodiment of the present invention is preferably introduced to the blood vessel within a vessel supporting structure, for example including but not limited to a stent, blood clot filter, wire frame or stent-graft or the like. Optionally, the rotating portion is stably connected to the frame of a vessel support structure in a number of optional configurations, preferably including but not limited to radial installation within the vessel support structure, wherein the rotation portion is within the lumen of the vessel support structure. Optionally, the rotation portion may be coupled to the frame of the vessel structure wherein the rotating portion is optionally located extra-luminally, outside the lumen of the vessel support structure. Optionally, the rotating portion may be integrated within the plane of the vessel support structure's frame, preferably being continuously integrated within the frame of the support structure. The rotating portion's blades preferably provide the vessel support structure with additional structural stability while also preferably maintaining an open lumen, wherein the rotating portion blades clear any developing blockages from within the lumen.

The vessel supporting structure and the rotating portion according to the preferred exemplary embodiment of the present invention is preferably expandable from a low-profile compressed condition to a larger profile expanded condition, wherein the resilient material urges the vessel supporting structure to expand radially, and to thereby apply radial force against the blood vessel's inner wall surface.

The preferred embodiments of the present invention comprising the vessel support structure and the rotating portion may preferably be composed of metallic or plastic material including a shape memory alloy (SMA) such as but not limited to nickel titanium alloy (NiTi) also known as nitinol having transition temperature around body temperature or optionally a shape memory polymer (SMP) that can be triggered in response to changes in heat, pH, electric or magnetic fields. For example, the device of the preferred embodiment of the present invention can be introduced into a blood vessel in its collapsed formation having a small profile. Once in place and after it is released from the constraining catheter, the device expands to the appropriate diameter and into its final or “memorized” shape.

Optionally and preferably the rotating portion, according to a preferred embodiment of the present invention, is capable of axial flow as an axial flow rotating portion. More preferably, the rotating portion is an across-flow rotating portion. Of course any type of rotational direction may optionally be implemented. Such a rotating portion is preferably associated with the vessel support structure and is preferably smaller than the diameter of the vessel support structure's lumen. Optionally at least one rotating portion may be present within the diameter of the lumen. Optionally and preferably a plurality of rotating portions may be placed sequentially and incrementally, in a step like manner, to diagonally span the diameter of the vessel support structure. Optionally a plurality of rotating portions may so be placed wherein any cross section angle, including horizontally or vertically, is covered.

The optional, exemplary cross flow rotating portion preferably comprises a plurality of blades that are optionally shaped like a helical or skewback airfoil. These blades are optionally and preferably oriented transversely and perpendicularly to the fluid flow and parallel to the axis of rotation. Optionally, the blades have hydrofoil sections that provide tangential pulling forces in the cross fluid flow, allowing the forces to rotate the rotating portion in the direction of the leading edge of the blades. Therefore the direction of rotation of the rotating portion preferably depends significantly, and more preferably only, on blade orientation, rather than on the direction of fluid flow.

Preferably the blades of this embodiment of the present invention are helical airfoils that are warped into a spherical or elliptic shape. Optionally, the blades may have variable widths along the blade and/or its shaft. Optionally, a different combination of blade types may be used for an individual rotation portion. The orientation and shape of the rotating portion's blades preferably allow the rotating portion to be a self-starting rotating portion, such that rotation is preferably initiated upon initiation of blood flow and such that the blades rotate even in very slow blood flow.

Optionally and preferably, the shape and dimensions of the rotating portion may be controlled and adjusted to best accommodate one or more of vessel geometry and flow rate (and pressure). Optionally, the various components comprising the rotating portion, for example preferably including the blades and anchors, may be controlled and adjusted to best accommodate one or more of vessel geometry and flow rate (and pressure).

Optionally, the rotating motion and speed of the rotating portion may be optionally controlled. Optionally rotation motion may optional be gained from external sources for example including but not limited magnetic energy induction. The blades of the rotating portion structure according to this optional embodiment may optionally be composed or integrate permanent magnets or optionally the blades could be produced of or coated with magnetic material. In another embodiment, the rotating portion control is accomplished by placing an invasive endoluminal electrical cables connected to conductive windings incorporated within a vessel supporting structure. Thus, producing rotation of the rotating portion in similar way to the rotation of electrical engine. Furthermore, according to this optional embodiment to external sources control and stimulus may be utilized to recover the rotating portion operation in case of body tissues build-up interfere with the rotating motion.

According to some embodiments, optionally windings are incorporated with the device of the present invention. Optionally, windings are an electrical wire structure that preferably takes the form of a continuous open loop, for example including but not limited to a solenoid, or solenoid like structure. Preferably, windings are subjected to a changing magnetic field that induces electrical flux, thereby creating an electrical current.

It should be noted that the rotating portion only harvest the hydrokinetic energy of the rotating portion sectional area through which the fluid passes. The shape and number of blades of the preferred non limiting embodiment of the present invention, optionally provides the rotating portion with efficiency control.

One of the many distinct advantageous of the preferred embodiment of the present invention is that the rotating portion can be designed by adjusting the blades properties to a specific efficiency in order to allow the fluid to flow freely, essentially not interfering with blood flow and not damaging the blood cells.

The shape, number of blades, size and overall construct of the rotating portion blades of the preferred embodiment of the present invention, including but not limited to a spherical or elliptic form, may be designed to customize and control the efficiency of the rotating portion including but not limited to the energy conversion efficiency of the rotating portion. A non-limiting example of how rotating portion blade design to bring about efficiency control may be defined by the following equation,

ε=Rotating portion efficiency (average)*Dt²/Dv²

where ε is the harvested energy percentage, Dt is the rotating portion diameter (mm) through which the blood hydrokinetic energy can be harvested, Dv is the inner walls vessel diameter (mm).

The blades are optionally mounted with at least one supporting members, which may optionally be mounted onto a rotatable shaft or a fixed shaft supported by at least one lightweight structure. The blades attached to an axis or shaft produce rotational spin about the axis creating rotational mechanical energy that may optionally be utilized to clear the lumen of the vessel support structure of any accumulated plaque. Optionally, the mechanical energy produced by the rotating portion of the present invention may be coupled to an electronic portion to produce electrical, magnetic, optical, electrostatic and RF energy or other forms of useable and preferable transmittable energy. More preferably the electronic portion may be integrated with the rotating portion of the present invention allowing the rotating portion blades to serve as the generator “rotor” and the “stator” windings to be incorporated within a vessel supporting structure including but not limited to a stent like structure, stent-graft, filter, wire frame or other like vessel support structure. The windings should be coated or covered with is electrical isolated material in order to isolate the generated electricity from the body. This optional configuration allows miniaturization of the rotating portion structure into miniature scale.

The blades of the rotating portion structure according to the preferred embodiment may optionally be composed or integrate permanent magnets on their external surface or optionally the blades could be produced of or coated with magnetic material.

The vessel support structure, the rotating portion and the filter portion in accordance with a further optional preferred embodiment of the present invention may produce electrical energy (electric potential), or electrostatic energy by the addition of metals and/or polymers that are naturally charged, or, alternately, by incorporating piezoelectric materials which may generate electric potential. Such metals, polymers and piezoelectric materials may generate electricity or electric potential by the rotating action of the rotation device that generated electric potential.

The power produced by an implantable energy harvesting and producing device such as the rotating portion system of a preferred embodiment of the present invention may optionally and preferably be transferable. A methods of safe energy transfer either in wired or wireless forms, as described in the present invention, is designed to ensure both power delivery and device safety as the energy transfer module is isolated from the device itself both receiving and transmitting in a safe manner. Accordingly a still further optional embodiment of the present invention is the delivery method of the produced energy to other devices using wired form when it is in the direct vicinity and wireless when it is not or needs to be electrically isolated. Isolating the power source is important in medical devices to prevent damage or incorrect operation of the powered device, for example electrical power surges may damage the circuitry or other delicate components due to over heating. Coupling provides significant protection preventing one circuit from adversely affecting another.

A still further optional embodiment of the present invention enables the conversion of the hydrokinetic energy of the blood into other forms of energy via the electronic portion of the preferred embodiment of the present invention. The forms of energy produced may take the form of at least one but not limited to electrical energy, radio frequency (RF) and optic energy. The use of the wireless forms of energy such as RF, heat and optic enables to keep the device and the medical device electrically isolated by converting electrical power to another form of energy optionally including but not limited to radio frequency (RF), heat and optic energy.

The converted wireless energy may be optionally directed to but not limited to a medical device located within the receiving distance of the wireless energy. Optionally, the transmitted wireless energy spreads/progresses in blood and tissues without severe energy attenuation. Optionally, the optic energy may be produced by an LED or laser diode that preferably produces light in the 820-840 nm range that is able to penetrate deeply into the tissue and is not absorbed by blood. Optionally wavelengths in a wider range such as 720-904 nm may be used to transmit optic energy. The wavelength used by the device depends on the tissue penetration depth that is required, similarly the type of light or laser source include but is not limited to Argon, HeNe, GaAlAs, Nd: YAG. For example, use of invisible infrared light (880 nm) has been shown that it can penetrate to a depth of about 30-40 mm without strong absorption along the way. The penetration distance is then governed by the wavelength of the light used.

To allow the transferred optic energy to be used, the destination and source preferably has a mechanism for converting the optic energy back to electrical energy, optionally including but not limited to a photoconductor.

Preferably the rotating portion and the electronic portion may be fitted within the vessel support structure. Optionally the rotating portion and the electronic portion may be fitted on the surface of the vessel support structure. The rotation portion may be fitted on the external surface of the vessel support structure, the internal surface of the vessel support structure or fitted within the vessel support structure or any combination thereof

The device or one of its components according to any one of the embodiments of the present invention may optionally be retrievable. For example, a recovery sheath can be delivered over the guide wire using over-the-wire techniques to collapse the expanded device for removal from the patient's vasculature as known in the art. Alternatively, for example, following the completion of the therapeutic or diagnostic procedure, an angioplasty catheter with distal introducer, can be used to retrieve a filter device by withdrawing the guidewire proximally into the distal introducer and pulling the filter device into the distal introducer.

A further feature of the present invention in any one of its embodiments is the ability to retrieve one of its components. The rotating portion and/or the electronic portion may optionally be retrieved by medical instrument or intervention. For example medical instrumentation able to retrieve the components may include but is not limited to an angioplasty catheter with distal introducer and retrieved back into the distal introducer in a manner similar to the other embodiments already described by using the locking mechanism of the guidewire to interact with the rotating portion and/or the electronic portion and pull the rotating portion and/or the electronic portion proximally either partially or completely into the introducer catheter.

A further exemplary embodiment for disabling or removing a component of the present invention is by using a non-compliant balloon that may be inflated against the vessel walls in order to compress the luminal components such as the rotating portion against the vessel wall. Optionally the rotating portion and electronic portion may then be securely detached and removed from an associated object including but not limited to vessel support structure, secondary object or the like with a mechanical assembly such as but not limited to turn lock assembly or a pin lock assembly. More preferably a component may be removed by taking advantage of the device's material properties that can optionally be triggered in response to changes in heat, cold, pH, electric or magnetic fields in the vicinity of the rotating portion and the electronic portion applied by external source. Optionally the rotating portion and/or the electronic portion may be controllably disabled or released with any combination of the methods described.

Optionally the device vessel support structure is shaped as an airfoil on internal surfaces of the vessel support structure for increasing blood flow through the rotating portion.

Optionally the device of the present invention in any of its embodiments comprising the vessel support structure, the rotating portion may preferably but optionally be composed of metallic material for example including but not limited to 300 series stainless steels, platinum, platinum-iridium alloys, cobalt-chromium alloys such as MP35N, unalloyed titanium, or the like.

Optionally the device of the present invention in any one of its embodiments may optionally in whole or in part may be composed of plastic or metallic material for example including but not limited to a shape memory alloy (SMA) such as but not limited to nickel titanium alloy (NiTi) also known as nitinol, having a transition temperature around body temperature or a shape memory polymer (SMP) that can optionally be triggered in response to changes in heat, pH, electric or magnetic fields. Optionally any other materials or any combination thereof may be used.

A still further optional non limiting embodiment provided by the present invention is an inherent blood flow rate sensor where the blood flow rate passing through the device is proportional to the rotating portion rotation speed. Optionally, this may serve as either a primary or secondary sensor for devices associated with the rotating portion of the preferred embodiment of the present invention including but not limited to a implanted cardioverter defibrillator (ICD) potentially minimizing the defibrillator's false alarm rate. This exemplary non-limiting embodiment may optionally communicate vital data regarding the flow rate, cardiac output and vessel occlusion state as inferred from the activity or inactivity of the rotating portion of the preferred embodiment of the present invention.

The device of the present invention in any of its embodiments may optionally be coupled to various objects and therefore may serve as a platform for introducing secondary or peripheral objects for example including but not limited to sensors, medicaments, small scale devices, or the like. Most preferably the secondary objects are coupled to the rotating portion of the device of the present invention preferably utilizing the rotational speed to control the secondary device. For example, the rotational speed of the rotating portion may optionally and preferably act as a trigger to release the medicament, electrical current, or to communicate sensed such as cardiac parameters for example including but not limited to blood pressure, flow rate, temperature, pH, or the like.

Optionally the device of the present invention, in any of its embodiments, may be coupled to a secondary object by various means. For example a medicament may be coupled to the device or any portion thereof by method including but not limited to loading, or coating or the like.

The device of the present invention, in any of its embodiments, may optionally serve as a platform for carrying at least one sensor for example including but not limited to physiologic sensor, hematological sensor biochemical sensor and like sensors. Preferably and optionally a sensor may be coupled to the rotating portion of the device of the present invention. The sensor will optionally enable continued monitoring of at least one parameter for example including but not limited to temperature, blood pressure, heart rhythm, pH, electrolytes, blood sugar, blood cholesterol levels or the like.

The device of the present invention in any of its embodiments may be coupled with a medicament for example including but not limited to blood thinning drugs, hormones, genes or the like. Most preferably the medicament is coupled to the rotating portion of the device. Optionally the device may be loaded with a medicament optionally by coating or by coupling small aggregates that release the medicament for example including but not limited to hormones, genes or the like. Optionally the release of the medicament may preferably be controlled by the activity of the rotating portion of the device and optionally include automatic release, uniform release, reaction for example a magnetic reaction, rotating speed reaction (high or low blood flow rate), servomechanism, sensor programming or external control.

In any some of the embodiments of the present invention, the device may act as a foreign body that may stimulate the formation of thrombi or occluding material. Accordingly, the deployment of the device of the present invention may be coupled to a course of drug treatment, for example including but not limited to drugs that inhibit or control the formation of thrombus or thrombolytics such as heparin or heparin fragments, aspirin, coumadin, tissue plasminogen activator (TPA), urokinase, hirudin, and streptokinase, and other suitable therapies may be used.

The device of the present invention in any one of its embodiments may be optionally coated with a substance or structure that improves biocompatibility or tissue adaptation or coated for in vivo compatibility as is described and well known in the art. The device may be coated with one or more of the following: antiproliferatives (methotrexate, cisplatin, fluorouracil, Adriamycin, antioxidants (ascorbic acid, carotene, B, vitamin E, and the like), antimetabolites, thromboxane inhibitors, non-steroidal and steroidal anti-inflammatory drugs, Beta and Calcium channel blockers, genetic materials including DNA and RNA fragments, and complete expression genes, carbohydrates, and proteins including but not limited to antibodies (monoclonal or polyclonal) lymphokines, growth factors, prostaglandins, and leukotrienes, and other suitable therapies may be used.

The device of the present invention in any one of its embodiments may optionally in whole or in part be formulated or composed of biodegradable material, preferably reducing the risk of thrombus formation and reducing need for chronic implantation of vessel support structures. Optionally the biodegradable material may be composed of materials such as polylactic acid polyglycolic acid (PGA), collagenor other connective proteins or natural materials, magnesium alloys, polycaprolactone, hylauric acid, adhesive proteins, co-polymers of these materials as well as composites and combinations thereof and combinations of other biodegradable polymers, biodegradable glass, bioactive glass or the like. Preferably, biodegradable glass, bioactive glass is also a suitable biodegradable material for use in the present invention.

For example, the vessel support portion of the device may optionally be composed of biodegradable material wherein preferably over time the support structure is absorbed by the body after healing of the angioplasty site. Preferably by becoming absorbed into the vessel wall avoids the limitation of current vessel support structures such as a stent by alleviating the need for chronic implantation. Optionally and preferably it would be further desirable to use biodegradable material that could optionally be shaped in a desirable manner for example including but not limited to a mesh-like or porous configuration, that will optionally enable endothelial cells at the angioplasty site to grow into and over the vessel supporting structure so that bio-degradation will occur within the vessel wall. Similarly, any portion of the device may be formed with biodegradable matter for example including but not limited to the rotation portion, that may be degraded over time or optionally by a triggering event such that the biodegradation will only start upon a cue for example when the site is deemed to be healed. For example, in some implementation such as stent or stent-graft or an in-stent restenosis (ISA) the rotating portion of the device may not be required beyond a certain period of time. Optionally in such implementation the rotating portion may be composed of biodegradable material to minimize the risk of thrombus formation and other complications.

Also it should be emphasized that although the described embodiments refer to blood vessels and blood flow, the present invention may also optionally be employed within other lumen(s) of the body, preferably those lumen(s) through which there is fluid flow, including but not limited to the cerebrospinal fluid system, the lymphatic system, the gastrointestinal tract, the kidneys and bladder (urinary tract and associated system), the male reproductive tract, fluid flow within the eyes and so forth.

Unless otherwise defined, all technical and scientific terms used herein have the same meaning as commonly understood by one of ordinary skill in the art to which this invention belongs. The materials, methods, and examples provided herein are illustrative only and not intended to be limiting. Implementation of the method and system of the present invention involves performing or completing certain selected tasks or steps manually, automatically, or a combination thereof. Moreover, according to actual instrumentation and equipment of preferred embodiments of the method and system of the present invention, several selected steps could be implemented by hardware or by software on any operating system of any firmware or a combination thereof. For example, as hardware, selected steps of the invention could be implemented as a chip or a circuit. As software, selected steps of the invention could be implemented as a plurality of software instructions being executed by a computer using any suitable operating system. In any case, selected steps of the method and system of the invention could be described as being performed by a data processor, such as a computing platform for executing a plurality of instructions.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in order to provide what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1A-C are schematic diagrams of an exemplary miniature rotating portion according to an optional embodiment of the present invention; and

FIG. 2A-C are schematic diagrams of an exemplary miniature rotating portion according to an optional embodiment of the present invention; and

FIG. 3A-C are schematic diagrams of an exemplary miniature rotating portion according to an optional preferred embodiment of the present invention; and

FIG. 4 is a schematic diagram of an exemplary mounting of the miniature rotating portion according to an optional embodiment of the present invention; and

FIG. 5A-E are exemplary schematic diagrams of the blades used in the rotating portion according to an exemplary embodiment of the present invention; and

FIG. 6A-G is an exemplary schematic diagram of the vessel supporting structure and the rotating portion according to an exemplary embodiment of the present invention as implanted in a blood vessel; and

FIG. 7 is an exemplary schematic diagram of an implanted vessel supporting structure encasing a rotating portion and an electronic portion according to an optional embodiment of the present invention coupled to a device in accordance with an optional system of the present invention; and

FIG. 8A-C are exemplary schematic diagrams of an implanted vessel supporting structure encasing a rotating portion and an electronic portion; and

FIG. 9A-B are exemplary schematic diagrams of an exemplary embodiment of the present invention using energy conversion to transmit power between the device of the preferred embodiment and a medical device; and

FIG. 10 is a block diagram of an exemplary system according to a preferred embodiment of the present invention; and

FIGS. 11A-C and 12A-C depict an alternative optional mode of disabling and spreading the rotating portion of the preferred embodiment of the present invention; and

FIG. 13 depicts an optional mode of device delivery and realigning of the preferred embodiment of the present invention; and

FIG. 14A-C are exemplary schematic diagrams of exemplary embodiments of the present invention based on rotating portion mounting; and

FIG. 15A-C are exemplary schematic diagrams of different views of placement of a plurality of rotating portion according to an exemplary embodiment of the present invention.

### DESCRIPTION OF THE PREFERRED EMBODIMENTS

According to preferred embodiments, the present invention is of a system and a method for a hydrokinetic rotating portion that is implanted in a blood vessel, preferably within a vessel support structure, including but not limited to a tubular structure, stent like structure, stent, Stent-Graft or filter structure, or wire frame or other like vessel support structure. The device may optionally be coupled to a number of devices having variable uses, including but not limited to generating and supplying power to implanted medical, providing a communication gateway to and from miniature and medical devices, and providing continuous monitoring and sensing of vessel blood flow rate.

The principles of the present invention may be better understood with reference to the drawings and the accompanying description.

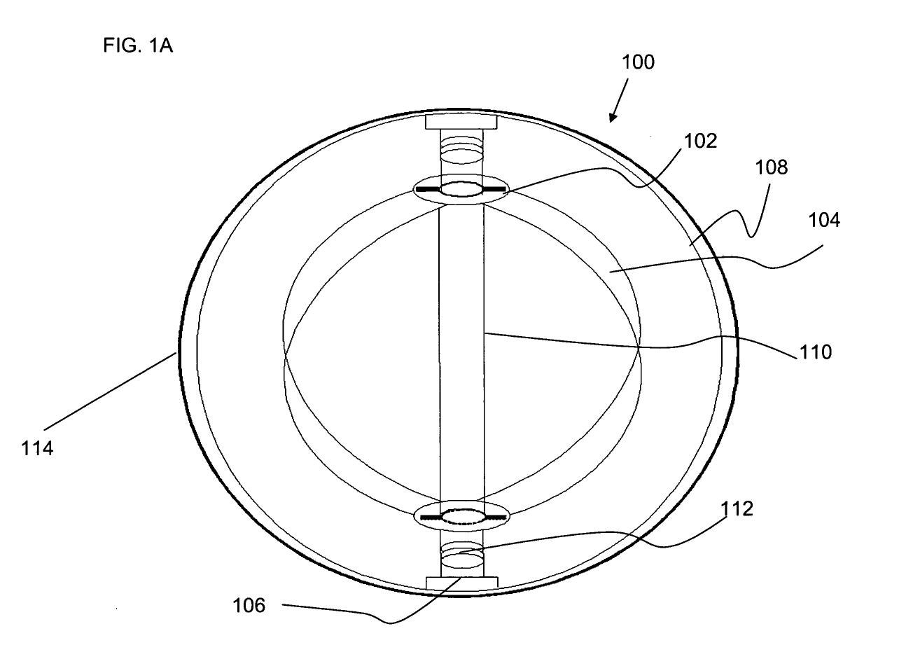

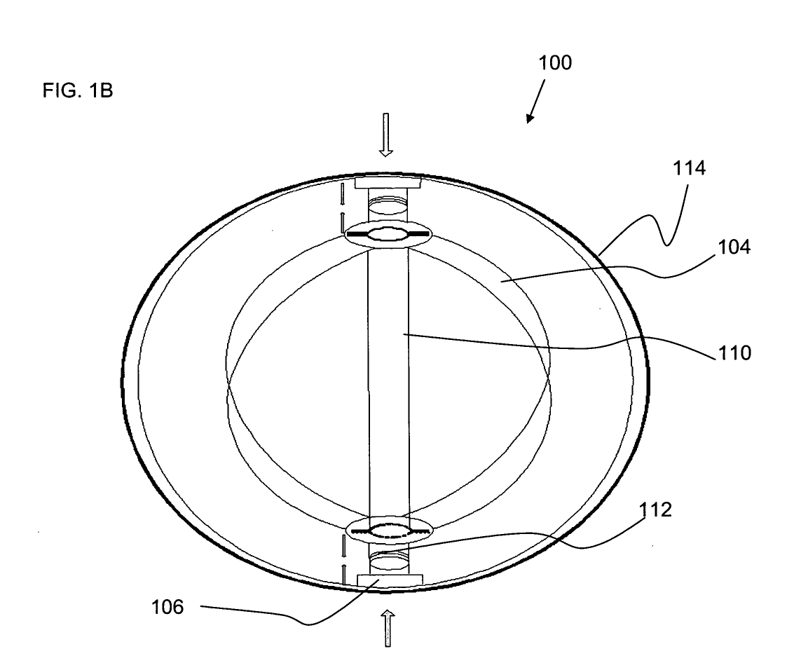

Referring now to the drawings, FIG. 1A is a schematic cross section diagram of an exemplary rotating portion 100 according to an optional embodiment of the present invention. Rotating portion 100 is attached to a vessel support structure 108, preferably including but not limited to a stent like structure, stent-graft, wire frame and other like vessel support structure, is inserted within the lumen of a blood vessel wall 114. Such insertion may optionally be performed according to stent introducing techniques known in the art, and incorporated herein by reference, by using a guiding catheter, a guide wire and a balloon angioplasty catheter. A stent delivery system that includes the stent that is advanced over the guide wire and the stent is then deployed at the site of the dilated stenosis.

Once in place within the blood vessel walls 114, the rotating portion 100 keeps the lumen open allowing blood to flow therethrough. Furthermore blades 104 preferably destroy any plaque or blockage material flowing through and accumulating in the lumen of vessel support structure 108. A rotating portion axis 110 spans the diameter of the vessel walls 114, and is positioned transversely and perpendicularly to the direction of blood flow to produce rotation in a plane parallel to the direction of blood flow. Rotating portion 100 is preferably anchored to vessel support structure 108 by at least one anchor 106.

Blades 104 are preferably coupled to rotating portion axis 110 via rotors 102 optionally molded and/or integrally formed with blade 104 to create a uniform structure. Blades 104 are optionally and preferably shaped to maximize rotational speed or to provide a specific energy harvesting efficiency; more preferably blades 104 are helical airfoil-shaped and spherically or elliptically warped.

As the blood flows through the lumen of the vessel support structure 108, it causes blades 104 and rotors 102 to spin. The spinning blades 104 and rotors 102 that define the operation of rotating portion 100 with respect to keeping the lumen of vessel 114 free of potential blockages, as potential blockage material passes through the vessel support structure 108 rotating portion 100 breaks it up into smaller pieces allowing the body to digest or absorb it naturally. Furthermore, anchor 106 tends to increase overall stability of vessel support structure 108 with regard to vessel walls 114.

However, because vessel walls 114 are not static and immobile, rotating portion 100 and vessel support structure 108 are preferably sufficiently flexible to accommodate for the natural forces acting on vessel walls 114. Accordingly, at least one flexible support structure 112, optionally including but not limited to a spring like element, is integrated between axis 110 and anchor 106, that provide rotating portion 100 with the required flexibility, allowing it to size itself according to the changing shape of vessel walls 114 while providing support structure 108 with sufficient structural integrity.

An optional but preferred extent of flexibility of rotating portion 100 is shown in FIGS. 1b-c. Accordingly, rotating portion structure 100 is preferably able to adjust one or more dimensions in accordance with the size of blood vessel walls 114. Flexible support structure 112 and flexible blades 104 preferably provide the structure of rotating portion 100 with the desired flexibility, allowing the rotating portion 100 to reshape itself in accordance with the various forces acting on the vessel walls 114.

FIG. 1B is an exemplary depiction of how rotating portion 100 and vessel support structure 108 may optionally and preferably be reshaped (i.e. to have a change in at least one dimension) in accordance with vertical forces that act on the vessel walls 114. Flexible structure 112 and flexible blades 104 absorb the applied vertical force, and preferably adjust at least one dimension to condense and hence to reshape rotating portion 100 to appropriately fit vessel walls 114.

Similarly, FIG. 1C is an exemplary depiction of how rotating portion 100 may optionally and preferably be reshaped (i.e. to have a change in at least one dimension) in response to horizontal forces acting on vessel walls 114. Flexible support structure 112 and flexible blades 104 preferably absorb the applied vertical force and expand, thereby adjusting at least one dimension to reshape rotating portion 100 to better fit vessel walls 114. Thus, blades 104, optionally made of pliable material, also preferably change configuration to better fit vessel walls 114 as constriction forces are applied on blades 104 both vertically and horizontally. Rotating portion 100 is optionally made of pliable material, allowing rotating portion 100 to be reshaped numerous times.

Blades 104 are optionally and preferably made of pliable material, optionally including but not limited to magnetic material incorporated within the blades 104, and/or a magnetic coating on the outer surface of blade 104 which may optionally be used to generate electrical power. Optionally blades 104 and rotors 102 may be fitted with other energy harvesting materials and devices that would allow use of their rotational energy to create alternative forms of energy including but not limited to a piezoelectric semiconductor able to intrinsically produce electric energy.

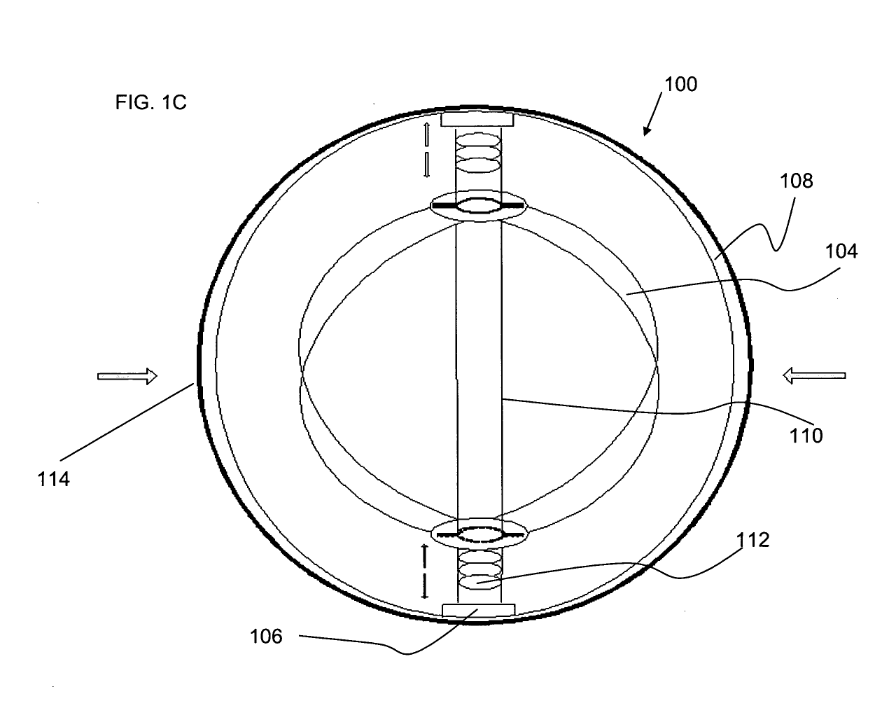

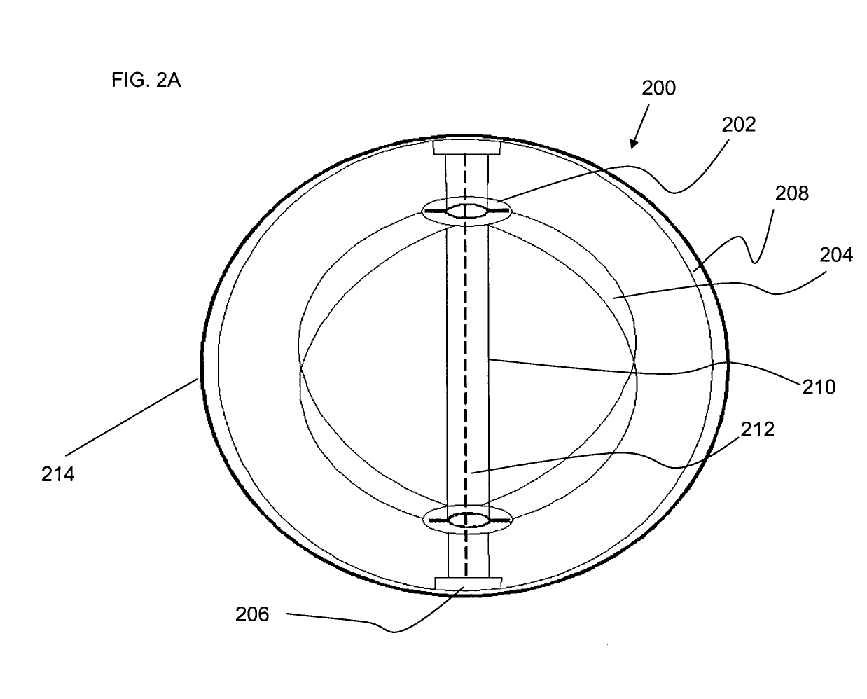

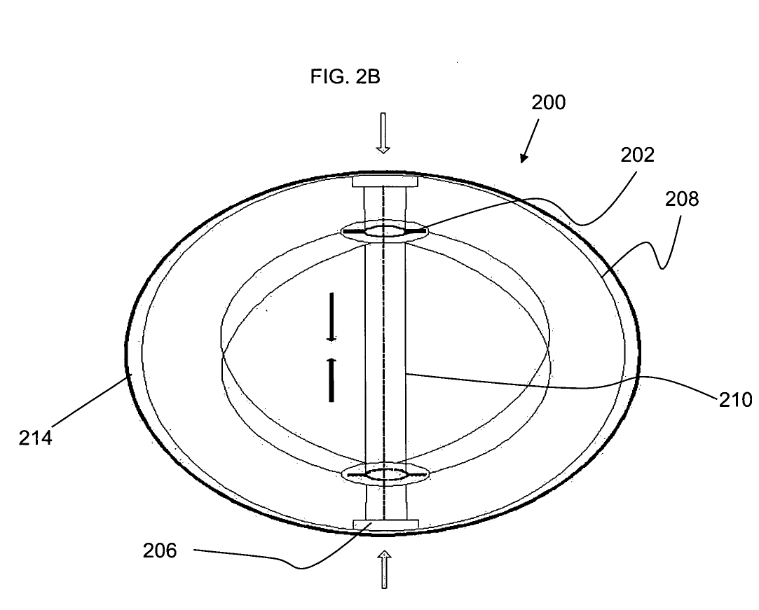

An additional embodiment of rotating portion 100 of FIG. 1 is shown in FIG. 2 that shows an optional, exemplary embodiment of the present invention. A rotating portion 200 is implanted within the lumen of a blood vessel wall 214, and as described above that is supported by a vessel support structure 208, preferably including but not limited to a tubular structure, a stent, blood clot filter, wire frame and stent-graft. A rotating portion axis 210 is preferably made of pliable or flexible material, and more preferably spans the diameter of the vessel support structure 208 lumen. Rotating portion axis 210 is also positioned transversely to the direction of fluid flow, producing rotation in a plane parallel to the direction of fluid flow. Rotating portion axis 210 is optionally and preferably held in position relative to vessel support structure 208 by at least one anchor 206. Along rotating portion axis 210 at least one and more preferably two rotors 202 are optionally molded with and/or integrally formed with at least one blade 204, that is optionally an airfoil-shaped spherically warped blade. As blood flows through the lumen of vessel support structure 208, it applies rotational forces on blades 204, causing them to spin and in turn spinning rotors 202. Axis 210 is preferably made from pliable material and provides rotating portion 200 with flexibility, allowing it to size itself (i.e. to change at least one dimension) according to the changing shape of vessel walls 214, as depicted in FIGS. 2B-C and also as noted previously with regard to FIG. 1.

Vessel walls 214 are not static, such that their shape is modified as constrictive forces are applied on them. Accordingly, rotating portion structure 200 is preferably able to resize itself (i.e. to change at least one dimension) in accordance with the size of blood vessel walls 214. Axis 210 preferably provides rotating portion structure 200 with the required flexibility and structure for such alteration of at least one dimension.

FIG. 2B depicts how the rotating portion may optionally and preferably reshape (i.e. to change at least one dimension) in accordance with vertical forces that act on the vessel walls 214. Axis 210 preferably absorbs the applied vertical force and condenses to reshape device 200 to fit vessel 214.

Similarly, FIG. 2C is an exemplary depiction of how rotating portion 200 may optionally and preferably reshape (i.e. to change at least one dimension) in response to horizontal forces acting on vessel walls 214. Axis 210 preferably absorbs the applied vertical force and expands to reshape rotating portion 200 to fit vessel walls 214 reconfigured shape. Furthermore, blades 204, optionally molded and/or integrally formed with rotors 202, are optionally made of pliable material and able to change configuration to fit the new shape with the vessel walls 214 as constriction forces apply to it both vertically and horizontally. Preferably rotating portion 200 is optionally made of malleable and pliable material allowing it to be continuously reshaped.

Blades 204 are optionally made of pliable material, including but not limited to a magnetic material incorporated within the blades and/or magnetic coating on the outside of blades 204, that optionally functions to generate energy. Optionally blades 204 and rotors 202 may be fitted with other energy harvesting materials or devices that would use their rotational energy to create alternative forms of energy including but not limited to a piezoelectric semiconductor able to intrinsically produce electrical energy.

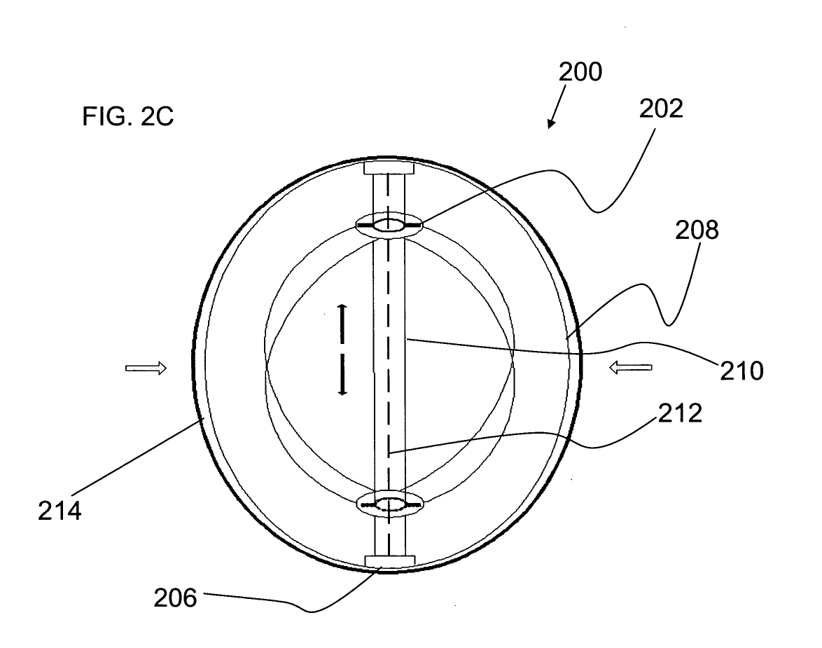

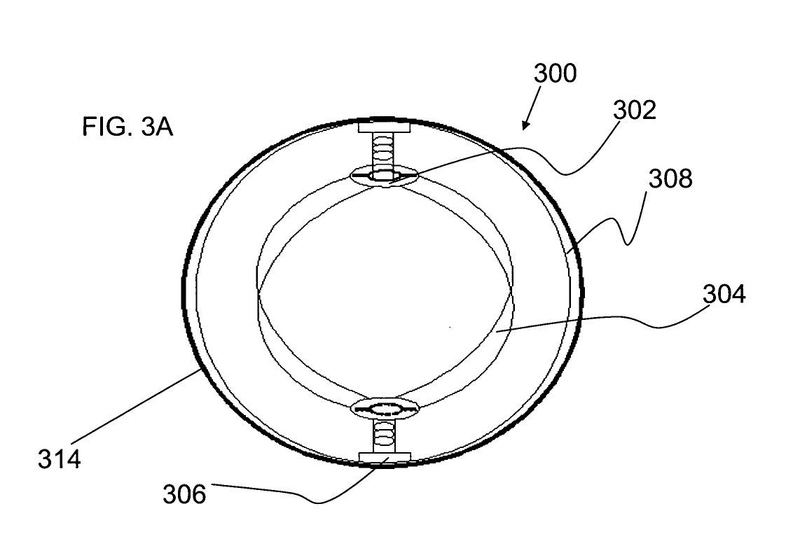

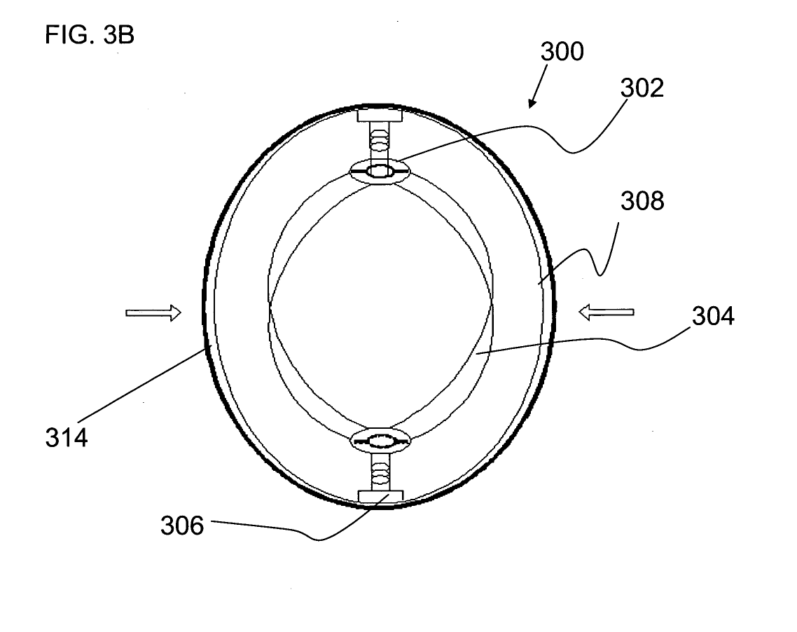

FIG. 3A presents a still further optional embodiment of the present invention that is another optional configuration of the rotating portion structure introduced in FIGS. 1 and 2 above. A rotating portion 300 is preferably implanted within the lumen of a blood vessel wall 314 supported by a vessel supporting structure 308, optionally including but not limited to a tubular structure, a stent, a coated stent, blood clot filter, wire frame and stent-graft. At least one support anchor 306 is positioned transversely to the direction of fluid flow to produce rotation in a plane parallel to the direction of blood flow, while securing rotating portion 300 in position relative to vessel support structure 308. At least one and preferably two rotors 302 are positioned and optionally molded and/or integrally formed with at least one blade 304, which is optionally and preferably helical and airfoil-shaped, and is more preferably spherically warped. As blood flows through the lumen of vessel support structure 308, it applies a force on blades 304 causing them to spin, and in turn this causes rotors 302 to spin. Blade 304, preferably made of pliable material, provides rotating portion 300 with flexibility allowing it to size itself (i.e. to change at least one dimension) according to the changing shape of vessel walls 314.

FIG. 3B is an exemplary depiction of how rotating portion 300 may reshape in response to vertical forces that act on the vessel walls 314. Anchors 306 preferably absorb the applied vertical force and condense (i.e. to change at least one dimension) to reshape rotating portion 300 to fit vessel 314.

Similarly, FIG. 3C exemplary depicts how rotating portion 300 may optionally and preferably reshape in accordance with horizontal forces acting on vessel walls 314. Anchors 306 preferably absorb the applied vertical force and expand to reshape (i.e. to change at least one dimension of) rotating portion 300 to fit a reconfigured shape of vessel walls 314. Furthermore, blades 304 preferably change configuration to fit the new shape with the vessel walls 314 as constriction forces act on blades 304 both vertically and horizontally. Preferably rotating portion 300 is optionally made of malleable and pliable material allowing it to be repeatedly reshaped.

FIG. 4 shows an optional non-limiting embodiment of rotating portion, with regard to mounting of the embodiment described in FIG. 1 above, such that rotating portion axis 110 (of FIG. 1) has been rotated 90 degrees to produce rotating portion axis 410 having a horizontal orientation. This rotation shows that a rotating portion according to any one of the embodiments of the present invention may be oriented in any manner within the vessel supporting structure 408 and blood vessel walls 414 as the shape of blade 404 determines the rotational direction.

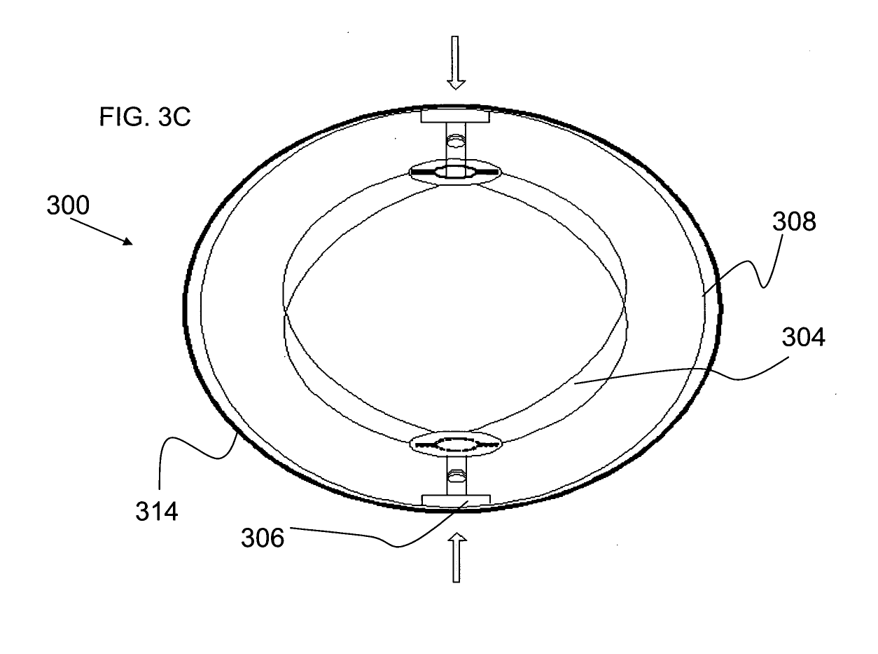

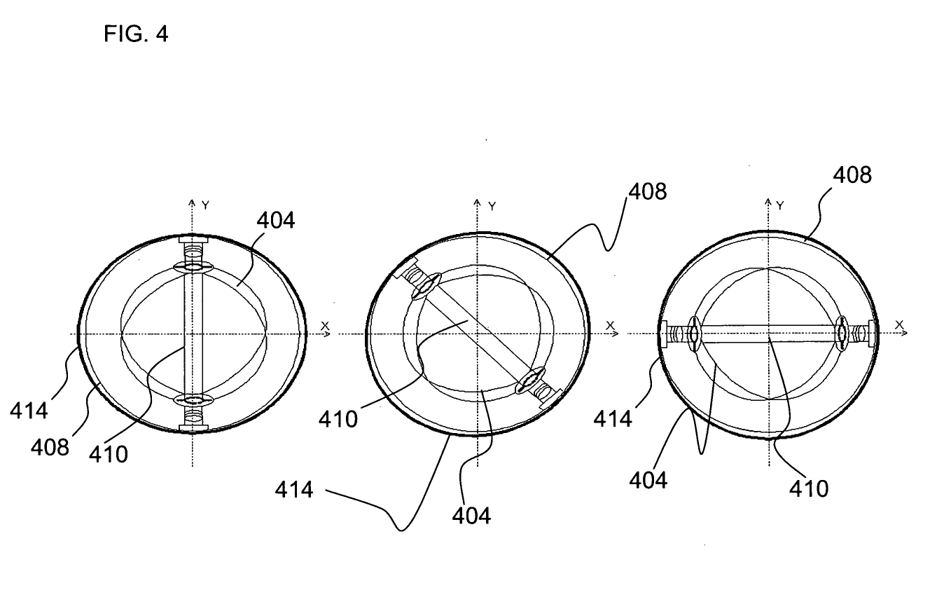

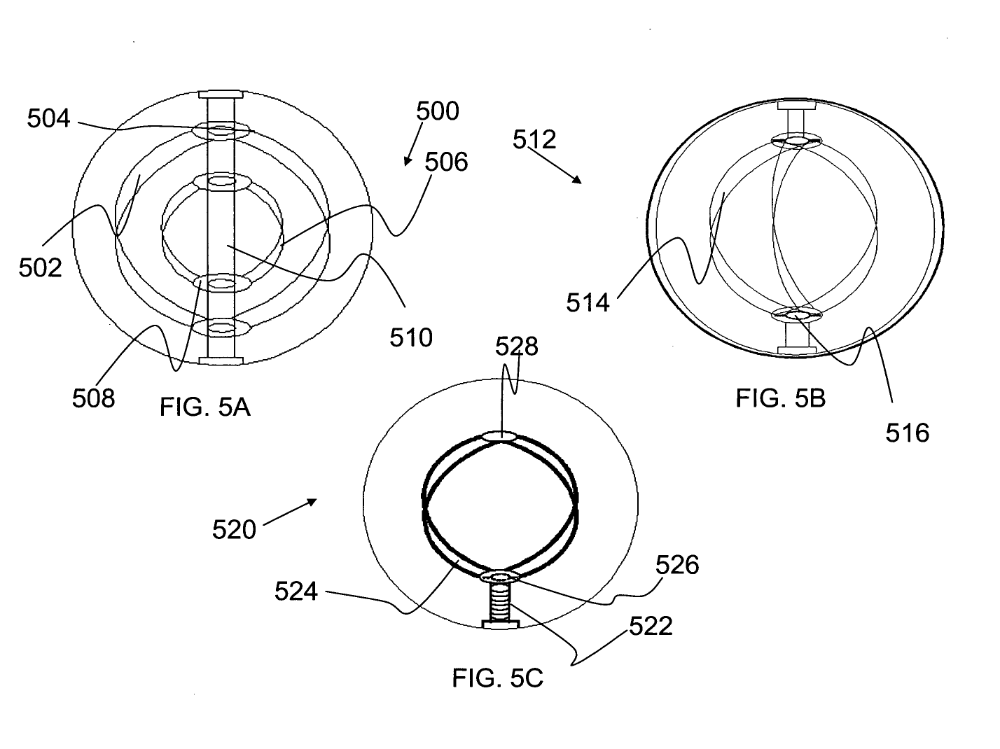

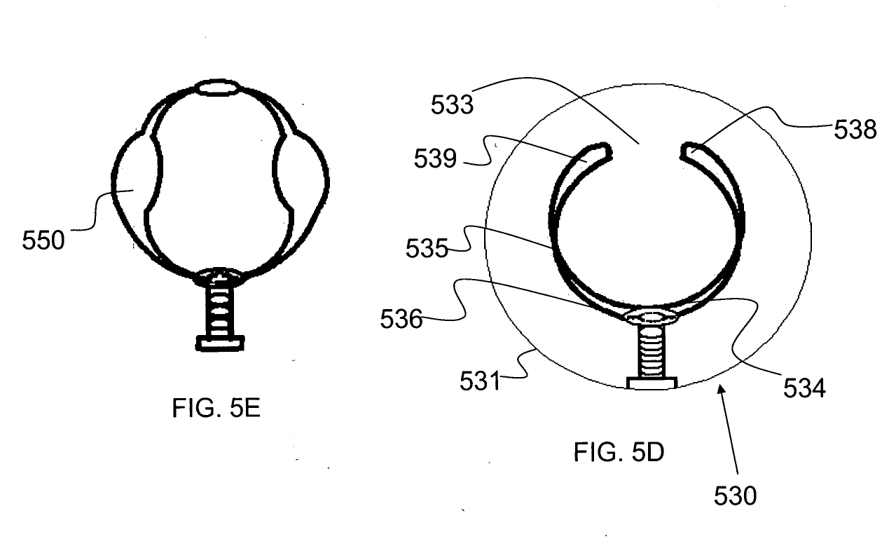

FIG. 5A is a depiction of a still further non limiting embodiment of the blades of the present invention, having an exemplary multiple layer configuration 500. Multiple layer configuration 500 preferably comprises at least two concentric blades as shown, an inner blade 506 attached to axis 510 via rotors 508 (optionally molded and/or integrally formed with inner blade 506), and an outer blade 502 optionally molded and/or integrally formed with rotors 504. The adjacent blades, inner blades 502 and outer blades 506, are preferably shifted circumferentially such that they do not overlap each other during rotation. That is, inner blades 506 preferably generate a spherical shaped rotating portion, which is positioned inside the outer spherical shaped rotating portion. The radius of inner blades 506 is preferably always smaller than the radius of the outer blades 502. The multilayer arrangement increases the torque of rotating portion 500.

FIG. 5B is a depiction of a still further non limiting embodiment of the rotating portion blades having a triple blade configuration 512. Triple blade configuration 512 preferably has three blades 514 that are optionally molded with rotor 516. The triple blade configuration may optionally be implemented with any rotating portion assembly configuration of a non-limiting embodiment of the present invention. Other multiple blade configurations having a plurality of blade groupings may optionally and preferably be implemented within the present invention.

FIG. 5C is a depiction of a still further non limiting embodiment of the rotating portion blades having one anchor configuration 520. Single anchor configuration 520 comprises one flexible anchor 522 and one rotor 526 that is optionally molded and/or integrally formed with at least one blade 524. Optionally, blade 524 is fitted with a flexible attachment 528 allowing a plurality of blades to be connected thereto. Anchor configuration 520 may optionally be implemented with any of the rotating portion assembly configurations previously presented according to the non-limiting embodiments of the present invention.

FIG. 5D is a depiction of a still further optional and non limiting embodiment of the rotating portion blades having a single anchor open configuration 530. Single anchor open configuration 530 preferably comprises one rotor 536 optionally molded and/or integrally formed with at least one blade 534. Upper tip 538 of blade 534 is open and preferably rounded to prevent damage to vessel wall 531, more preferably having a safety gap 533 from the upper tip 539 of another blade 535, for example to prevent a balloon angioplasty catheter from being caught between upper tips 538 and 539 of the rotating portion blades 534 and 535. The one anchor open configuration 530 may optionally be implemented with any rotating portion assembly configuration of a non-limiting embodiment of the present invention.

FIG. 5E is a depiction of a still further optional non limiting embodiment of the rotating portion's blades 550 having variable width along its length. Preferably the blades may be designed to produce the required rotational speed and momentum.

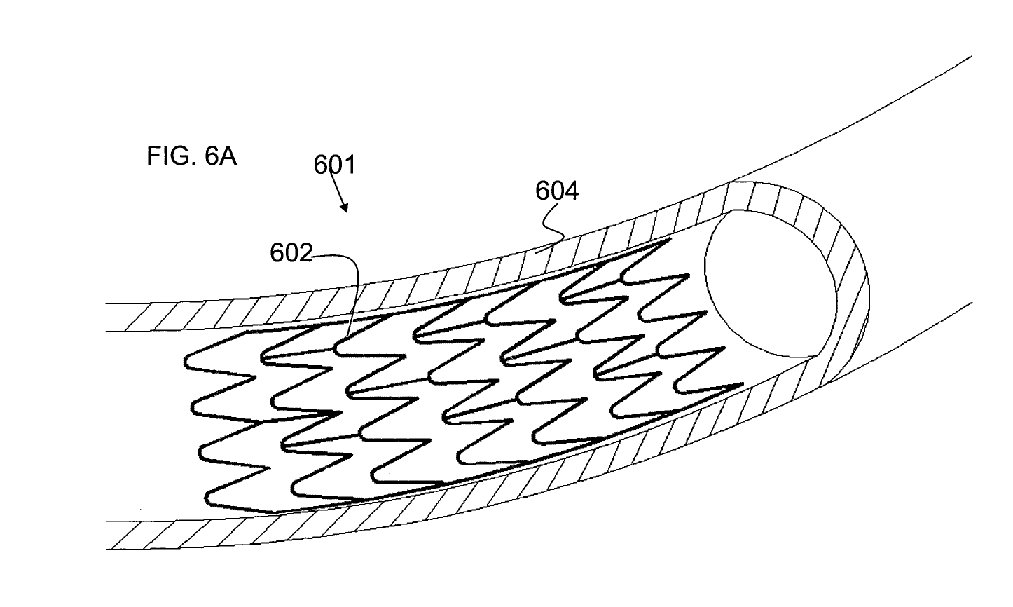

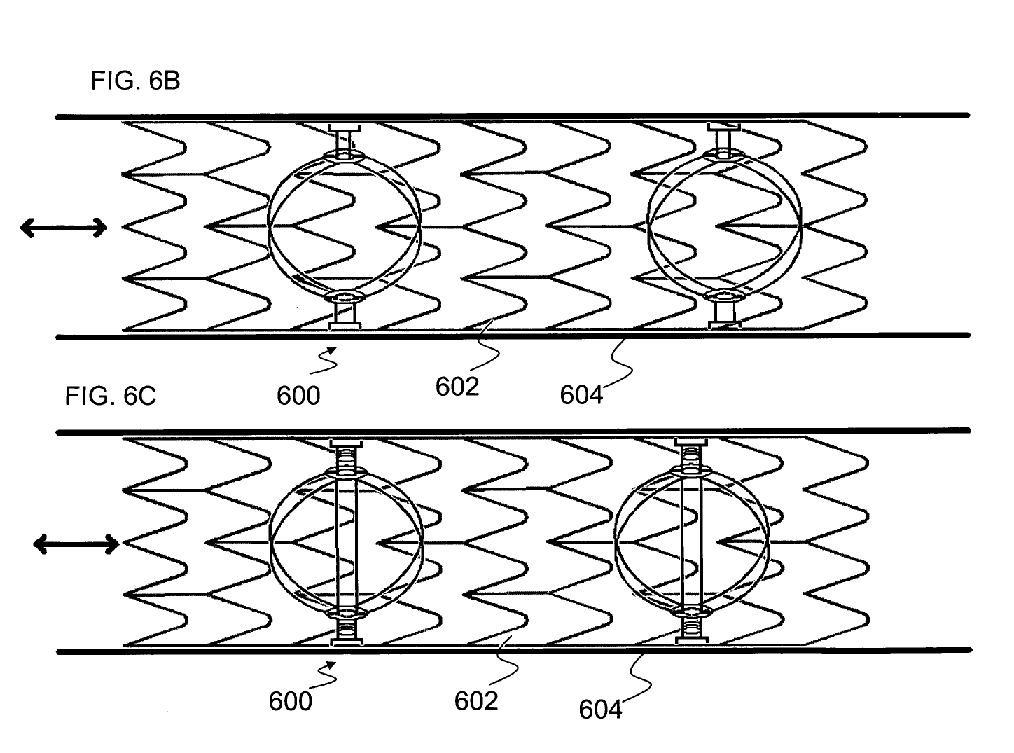

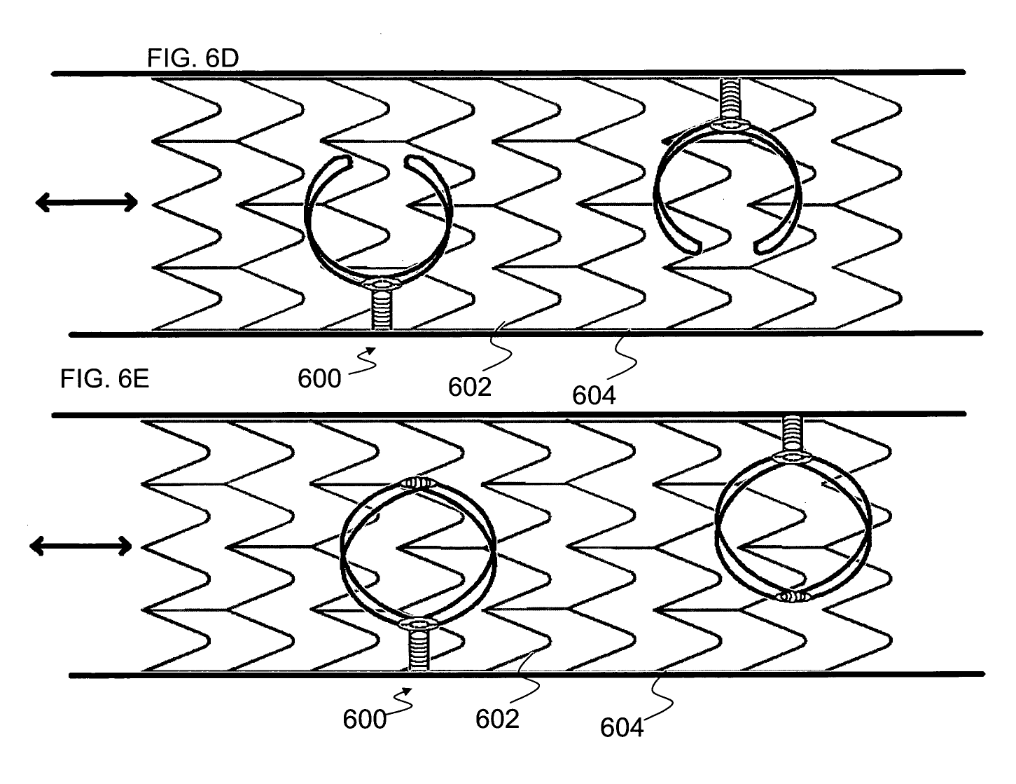

FIG. 6A is a cross-sectional view of a vessel supporting structure 602, which may optionally be implemented in any suitable form, preferably including but not limited to a tubular structure, a stent, blood clot filter, wire frame or stent-graft, imbedded in a blood vessel 601 having blood vessel walls 604. The vessel supporting structure 602 may optionally be fixed with a rotating portion structure (not shown). A rotating portion (not shown) according to the present invention preferably maintains both the structural integrity of blood vessel 601 while maintaining an open lumen allowing blood to flow freely through vessel support structure 602 as the rotating portion breaks down any plaques or other material that may form within or passing through vessel supporting structure 602.

FIG. 6B-G show an optional and exemplary implementation of the present invention, preferably comprising at least one rotating portion 600 according to any one of the rotating portion optional embodiments of the present invention as described in FIGS. 1-5 or any combination thereof in any orientation within a vessel supporting structure 602, which may optionally be implemented in any suitable form, including but not limited to tubular structure, a stent, blood clot filter, wire frame and stent-graft. Installation is within blood vessel wall 604 is preferably achieved by using a stent deployment method as is known in the art and incorporated herein by reference. Blood flows through vessel supporting structure 602 in blood flow direction 601, preferably causing the consecutive rotating portions 600 to spin, optionally and preferably generating mechanical power and while preventing plaque from forming within the lumen of vessel 600, vessel walls 604 or vessel support structure 602.

According to a preferred non limiting embodiment of the present invention, as rotating portion 600 spins it produces both electrical and mechanical energy. A number of rotating portions 600 may optionally and preferably be fitted into vessel supporting structure 602, each individually harnessing the hydrokinetic energy of blood flow in order to generate mechanical power, electrical power and other forms of energy.

The number of rotating portions 600 and/or their orientation in the vessel supporting structure 602 may optionally and preferably be varied to meet the various medical applications requirements and to enable free blood flow through the vessel supporting structure 602. For example, in order to harvest hydrokinetic energy uniformly and preserve the laminar flow of blood, the rotating portions 600 may optionally be oriented in such way that each rotating portion 600 receives flow from different volumes of the blood vessel 604 cross section. Thus, by preferably not allowing the same blood volumes to pass through more than one rotating portion 600, the hydrokinetic energy is harvested more uniformly.

Each rotating portion 600 may optionally be interconnected or function independently of the other. A greater number of rotating portions 600 housed within vessel support structure 602 is preferred as it provides a greater structural integrity.

FIG. 6F shows a system 606 featuring one or more rotating portions 600 that are able not only to spin but also to move vertically along and/or within vessel supporting structure 602.

FIG. 6G shows a system 608 featuring one or more rotating portions 600 that spin freely within the vessel support structure 610 and are enclosed by the vessel support structure 610.

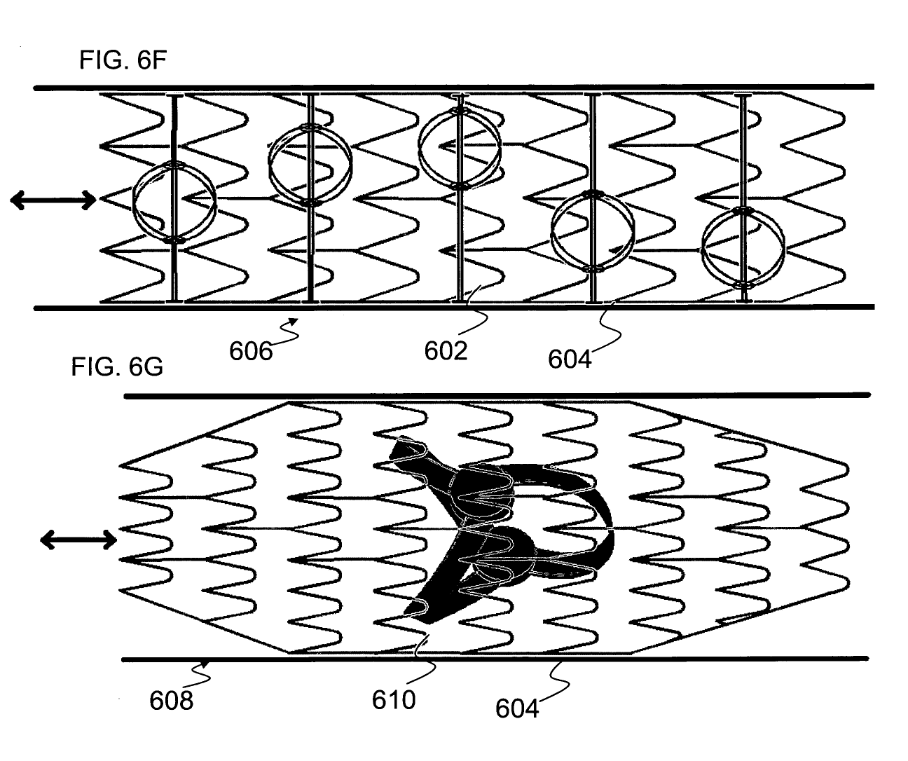

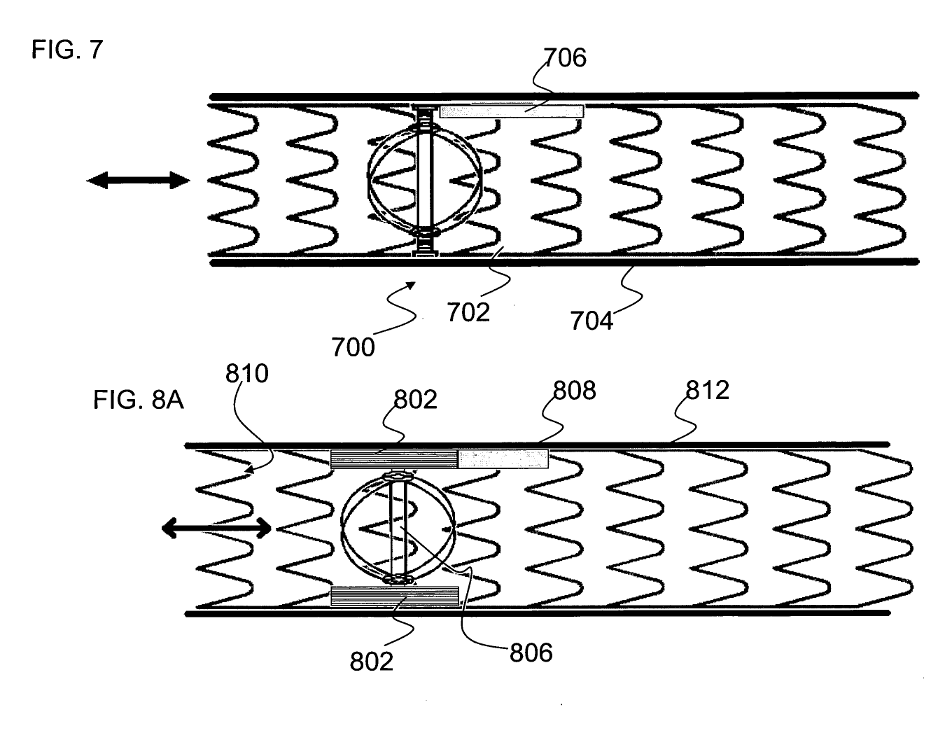

FIG. 7 shows a side view of an optional non limiting embodiment of the present invention where an exemplary rotating portion 700 is preferably attached to a vessel support structure 702 that is implanted in blood vessel walls 704, and is optionally coupled to device 706. The rotational mechanical energy created by rotating portion 700 optionally may be converted to electrical energy or other forms of energy, preferably by coupling rotating portion 700 to electronic portion 706 that may optionally include a generator, or optionally may itself be powered by the mechanical rotational energy produced by rotating portion 700.

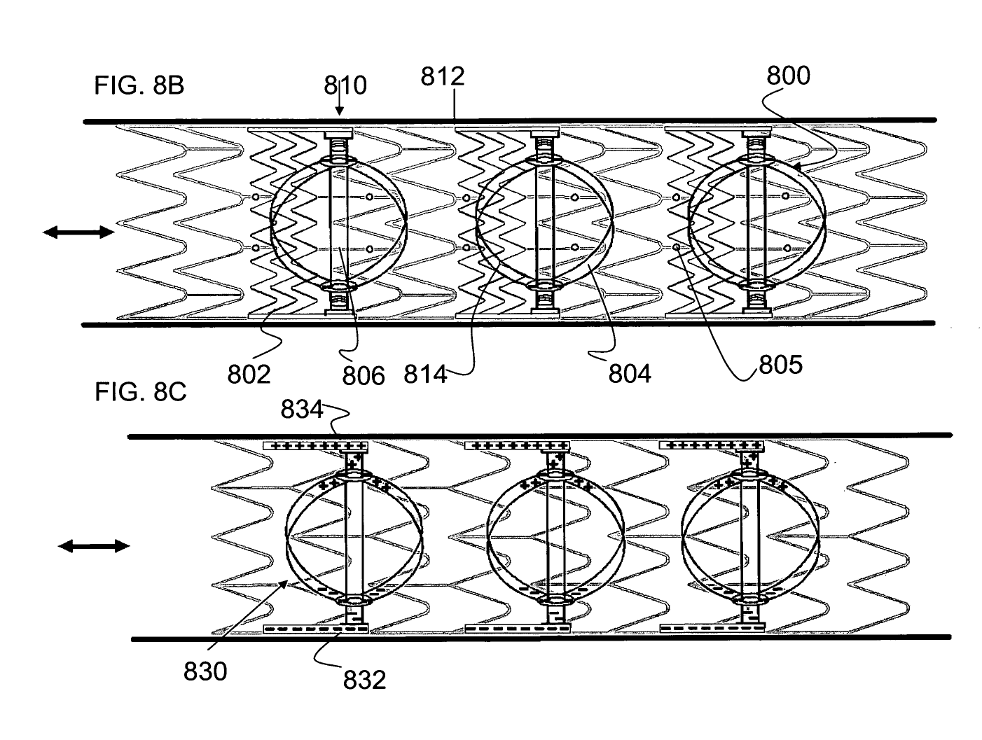

A still further exemplary embodiment of the present invention enabling the conversion of the mechanical energy produced by the rotating portion to electrical energy is depicted in FIG. 8. Blades 804 are optionally made of pliable material and optionally include a magnetic material incorporated within the blades 814 and/or a magnetic coating on the outer surface of blade 804.

Axis 806 is preferably incorporated in a vessel supporting structure 810 having windings 802 that are optionally conductive coils. Windings 802 are preferably arranged in a circumferential manner about blades 804, which may be optionally fitted perpendicularly relative to axis 806, as exemplified in FIG. 8B, or in parallel, as exemplified in FIG. 8A. The magnetic flux produced by the magnetic material 814 incorporated within the blades 804 and/or magnetic coating on the outer surface of blade 804 flows across the conductive windings 802.

When blood flows through support structure 810 housing rotating portion 800, it preferably causes blades 804 to spin, thereby causing permanent magnets 814 to rotate, so that a rotating magnetic field is formed about blades 804. Consequently, a magnetic flux crosses the stationary conductive windings 802 in alternately different directions, producing alternating current in each conductive coil 802.

FIG. 8B shows another embodiment of the windings 802 and rotating portion 800 that preferably comprise isolators 805, which function to isolate the body from the generated power. Windings 802 are themselves preferably isolated from the body and therefore electrical charge is not lost.

FIG. 8C shows a side view of an rotating portion 830 that is composed of chargeable material; therefore the spin of rotation portion 830 produces electric potential with positive charge 834 and negative charge 832. For example, the chargeable material comprising or incorporated with rotating portion 830 optionally includes but is not limited to metals, polymers or piezoelectric materials.

The voltage developed by the rotating portion 800 may optionally be calculated by the general equation:

∈=κ*N*S*ΔB/Δt

where κ=proportionality constant depending on the geometry and properties of the device; N=the number of wind conductive coils; S=the cross section area of the windings geometry; ΔB/Δt=total flux change over time (function of the angular velocity of the blades rotation).

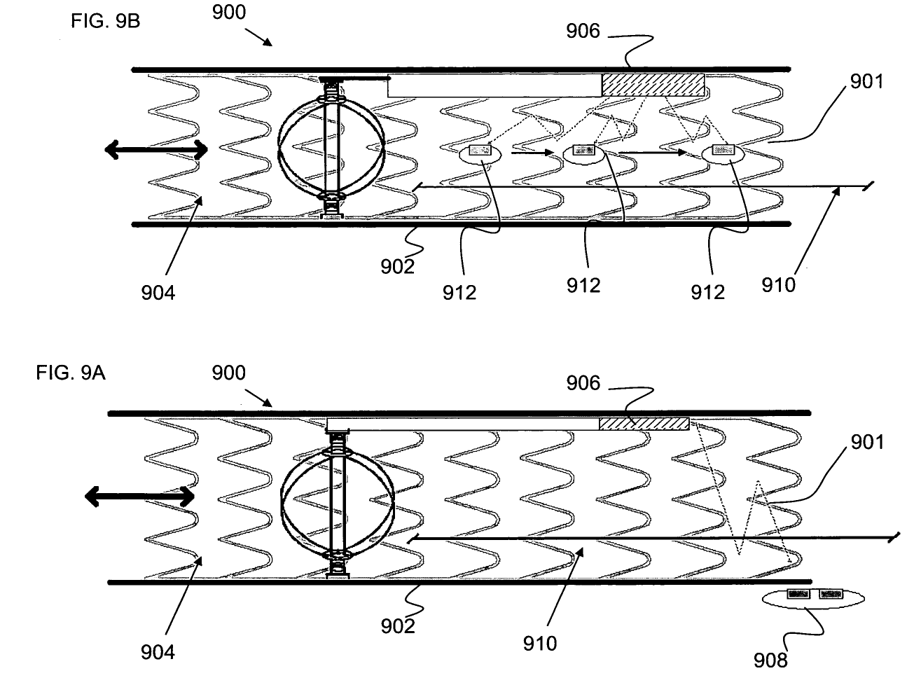

FIG. 9 displays a still further optional embodiment of the present invention where rotating portion system 900 is preferably located at a blood vessel wall 902 and is preferably housed within a vessel supporting structure 904; vessel supporting structure 904 optionally includes but is not limited to a stent, IVC filter, wire frame and stent-graft. Rotating portion system 900 is optionally fitted with an electronic portion. The electronic portion preferably comprises a power generator and an energy converter 906 that converts the electrical energy produced by rotating portion system 900 to optical energy such as but not limited to light in the infra-red and ultra-violet spectrum. The optical energy may optionally be transmitted to any device within an illumination zone 910, the area of which is specific to the type of optical energy produced by converter 906. Any device within illumination zone 910 may optionally use this form of energy.

Optionally and preferably medical device 908, shown in FIG. 9A, located outside of vessel walls 902, is able to receive and utilize the optic energy for its own intrinsic purposes. Similarly as depicted in FIG. 9B, a nano-scale medical device 912 located within vessel walls 902 and moving with the blood circulation preferably uses the optic energy in the illumination zone 910 in order to power and recharge a nano-scale device. Each time the nano-scale device passes through illumination zone 910 it able to recharge itself for operating outside the illumination zone 910.

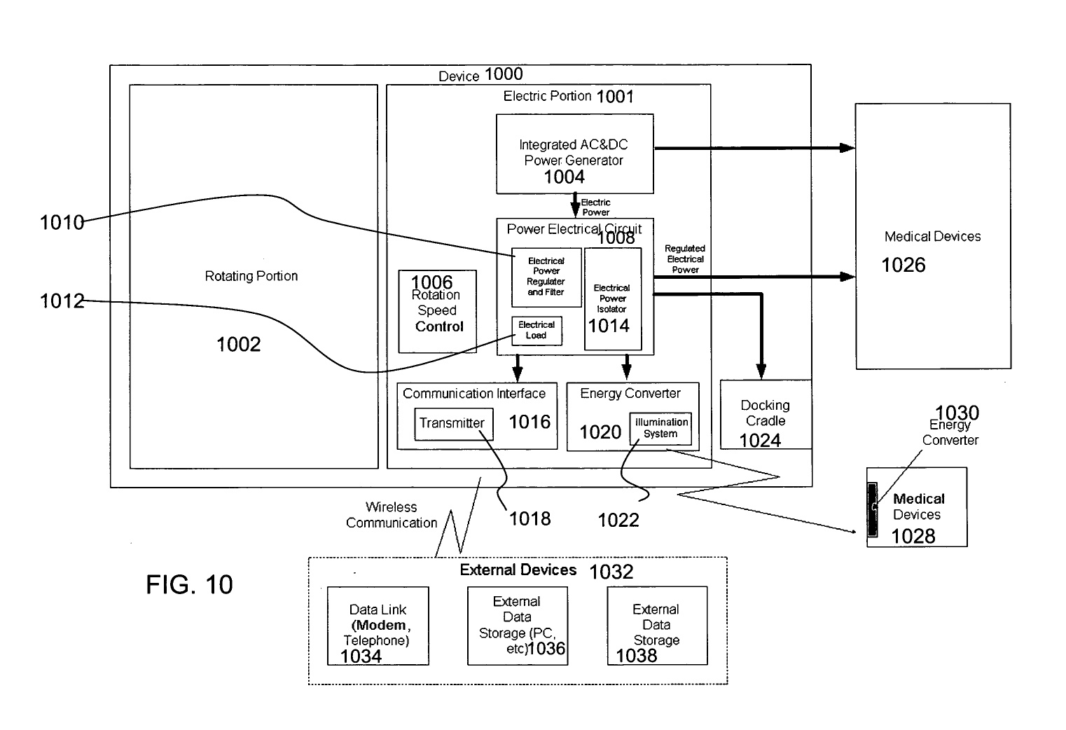

FIG. 10 is an exemplary non limiting block diagram of an optional system 1000 according to a preferred embodiment of the present invention comprising an electronic power circuit 1008; communication interface 1016;

energy converter module 1020; and docking station 1024. Rotating portion system 1002, optionally including but not limited to rotating portion 100 of FIGS. 1-3 and rotating portions of FIGS. 5A-5E. Electronic portion system 1001 preferably also comprises an AC/DC power generator 1004; optionally generator 1004 may take the form of AC/AC or AC/DC, converting the mechanical rotational energy of rotating portion 1002 to electrical energy. A rotating portion rotational speed control module 1006 preferably effectively controls rotational speed of rotating portion 1002 and therefore electrical production. There are a number of exemplary, illustrative methods to implement the rotational speed control module 1006 such as but not limited to controlling the shape and/or one or more other characteristics of the rotating portion's blades, providing traction control optionally molded with the blades, or induced magnetic force generated on the blades and opposite to the rotation direction (when the rotating portion blades are additionally optionally incorporated with ferromagnetic materials). Device 1000 may optionally produce at least one of the following forms of energy including but not limited to mechanical energy or electrical energy. Optionally the mechanical energy produced by rotating portion 1002 is directly coupled to a medical device 1026 that uses the mechanical energy for its own functioning. Medical device 1026 may optionally utilize the regulated electrical power produced by a circuit 1008 as a power source.

Optionally the electrical energy produced by generator 1004 may be coupled to an electric power circuit 1008 that functions to control the electrical energy. Electrical power circuit 1008 comprises an electric power regulator and filter 1010; electrical load module 1012 and power isolator 1014. Electric power regulator and filter 1010 preferably provide a smooth current output, optionally including but not limited to direct current and alternate current forms, therefore protecting system 1000 and any optionally associated devices from potentially damaging power surges.

Electric load module 1012 preferably provides internal load that dissipates the electrical power produced and not required by system 1000 or any devices or component coupled thereto, including but not limited to medical devices 1026 or 1028, communication module 1016, energy converter 1020 and docking station 1024. Electric load module 1012 is an optional module and may optionally be used as a secondary fail safe mechanism to the intrinsic control available to electrical portion 1001 preferably rotational speed control module 1006.

Optionally circuit 1008 also supports output voltage level calibration to allow for varying medical devices to be associated each having different power requirements. Optionally, the regulated electrical power produced by circuit 1008 may used by at least one of but not limited to an internal power source for system 1000, a medical device 1026, communication module 1016; energy converter 1020 or docking station 1024.

Communication interface 1016 preferably incorporates a transmitter 1018 allowing communication with external devices 1032, which may optionally include but are not limited to one or more of a modem data link 1034, a computer 1036, memory (not shown) or data storage device 1038 or medical devices such as cardioverter defibrillator. Transmitter 1018 optionally communicates either via a wired link or more preferably a wireless link and allows data to be transferred effectively making system 1000 a sensor that can transmit any data to the external environment, preferably obtaining medically derived data directly or indirectly from system's 1000 components. For example rotating portion 1002 may optionally and preferably reveal pertinent medical data including but not limited to blood flow rate, blood pressure, vessel disease status. Optionally communication module 1016 could transmit a special failure signal in order to indicate any system 1000 failures.

Docking station 1024 is optionally a station where other nanoscale devices may physically connect to system 1000. Preferably docking station 1024 allows the objects connected thereto to utilize the energy produced by device 1000 to recharge their intrinsic power sources, including but not limited to a battery or capacitor. Optionally by physically connecting to system 1000, the connection permits its components such as communication module 1016 to optionally be used, for example as a communication gateway, allowing the linked device to communicate via module 1016. The physical connection into docking station 1024 may optionally be undertaken by implementing an illumination guidance system including but not limited to a system specific signature wavelength or an interface for mutual identification.

Energy converter 1020 preferably converts the electrical power source of circuit 1008 to other forms of energy including but not limited to RF and optical energy, and more preferably comprises an illumination system module 1022. A preferred embodiment of the present invention allows the power produced by system 1000 to be converted to forms other than mechanical rotational energy or electrical energy. A non limiting form of energy that may optionally be used by system 1000 is optical energy, which is preferably transmitted to medical device 1028 located near system 1000, but not attached or otherwise directly linked to it. To be able to use the energy produced by device 1000, medical device 1028 is preferably located within the effective illumination zone of device 1000; therefore, optionally medical device 1028 may be located outside the blood vessel or location wherein device 1000 is implanted. Medical device 1028 optionally and preferably comprises an intrinsic energy conversion module 1030 allowing it to utilize the produced optic energy as an effective power source. This form of energy transmission is particularly useful for non-stationary nanoscale devices, and is particularly safe as surge protection is achieved.

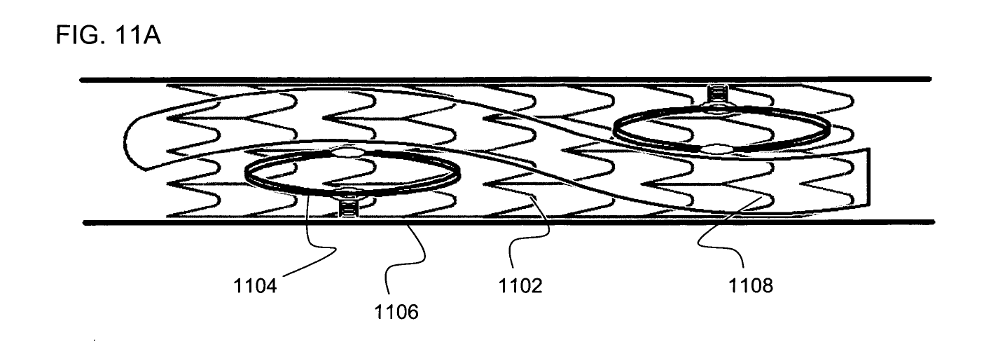

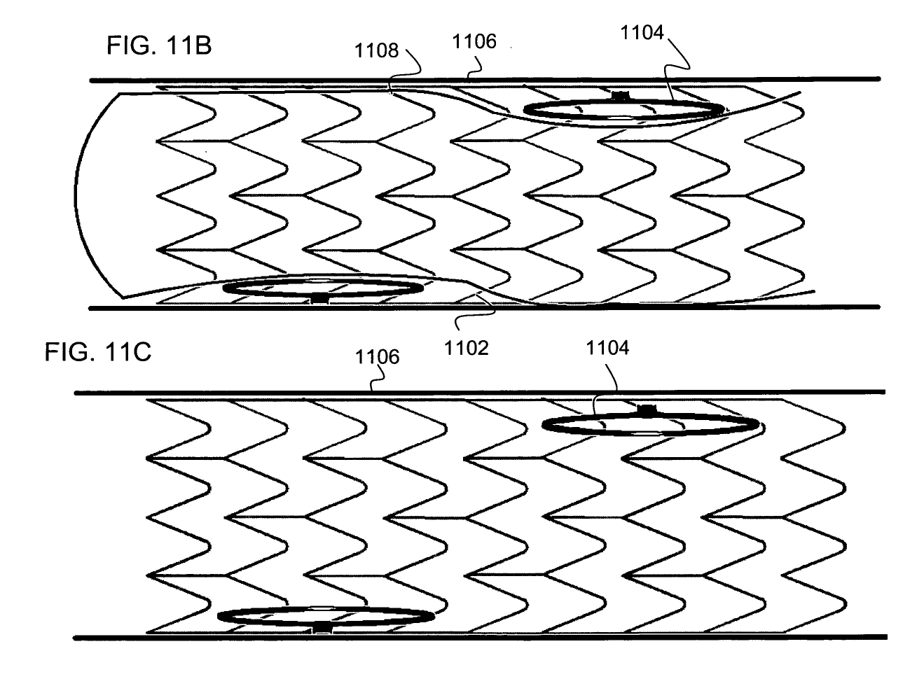

FIGS. 11 and 12 depict alternative optional modes of delivery of the preferred embodiment of the present invention, optionally using balloon angioplasty 1108 to guide device 1100 comprising vessel support structure 1102 and at least one rotating portion 1104 into vessel walls 1106. Following dilation of the blocked vessel using a guidewire catheter and balloon angioplasty 1101 as described in the art, device 1100 according to a preferred embodiment of the present invention is preferably deployed using a guidewire. Device 1100 is then expanded to fit vessel walls 1106 by inflating balloon 1108. Once device 1100 is securely in place, balloon catheter 1108 is preferably deflated and removed leaving device 1100 securely associated with vessel 1106.

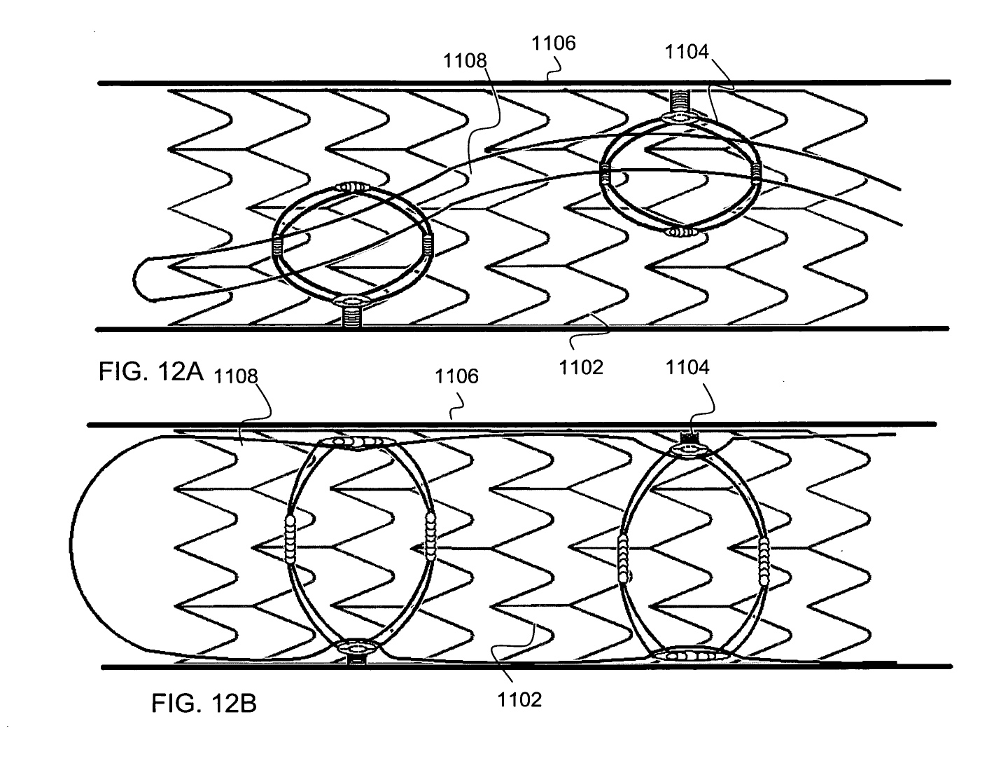

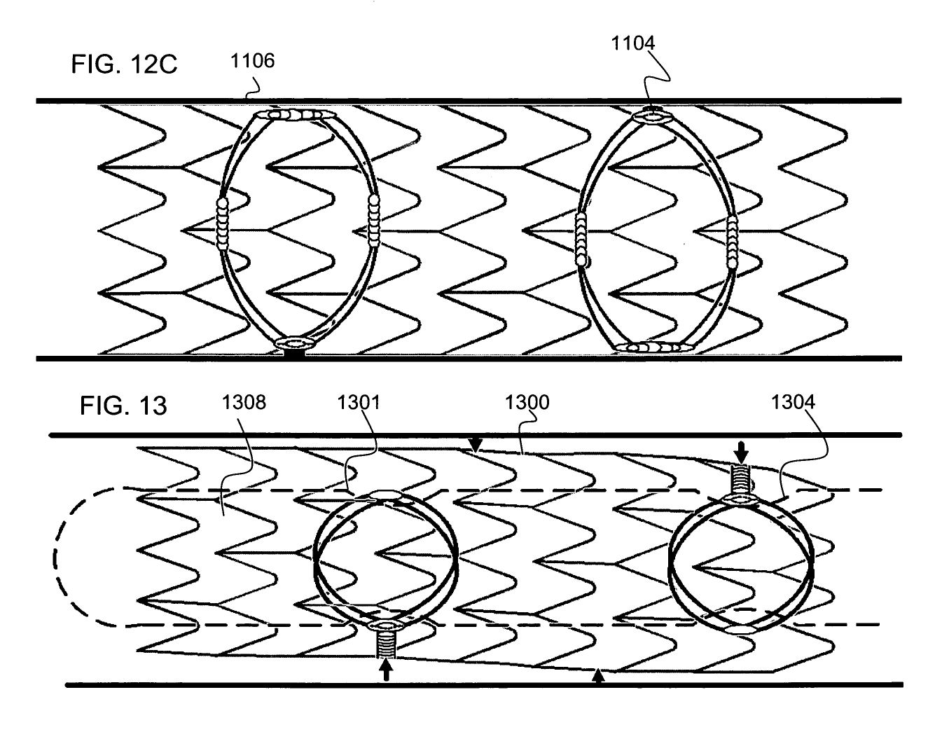

FIG. 13 depicts an alternative optional method of optionally realigning a shifted or misaligned vessel support structure 1300, comprising a rotating portion 1304 in accordance with any one of the preferred embodiments of the present invention. Vessel support structure 1300 is preferably realigned by using a grasping device 1301, optionally including but not limited to a hook. Grasping device 1301 is preferably associated with a balloon 1308 (for example from an angioplasty catheter), used to preferably realign or optionally remove support structure 1300. By partially inflating balloon 1308 and positioning the semi-inflated balloon 1308 at the center of the blood vessel lumen, the device profile decreases enabling the optional realignment or removal of vessel support structure 1300. Other grasping devices may be used additionally or alternatively.

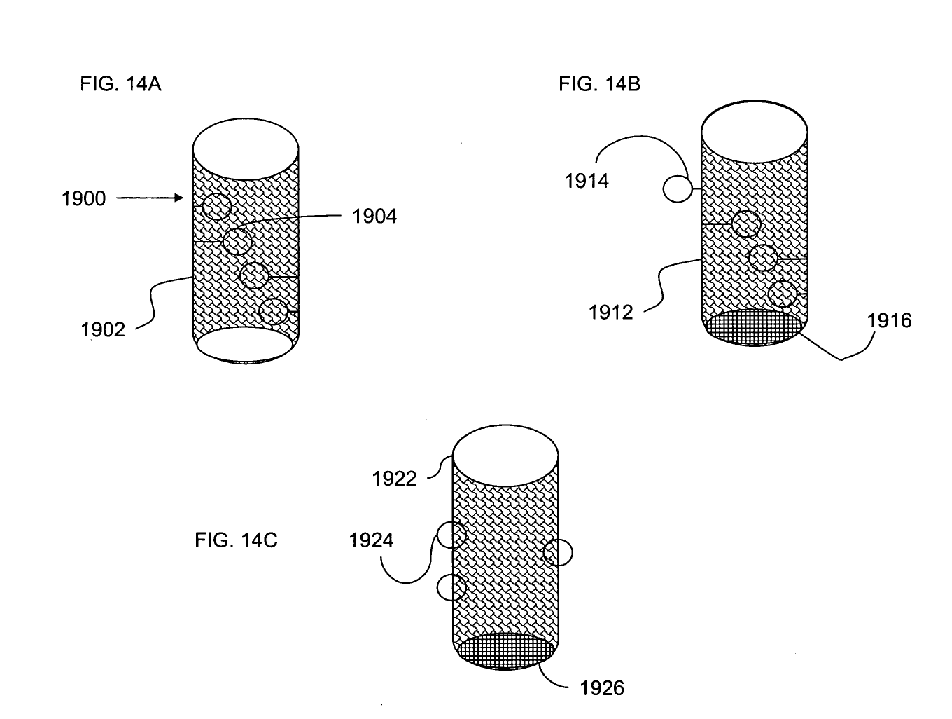

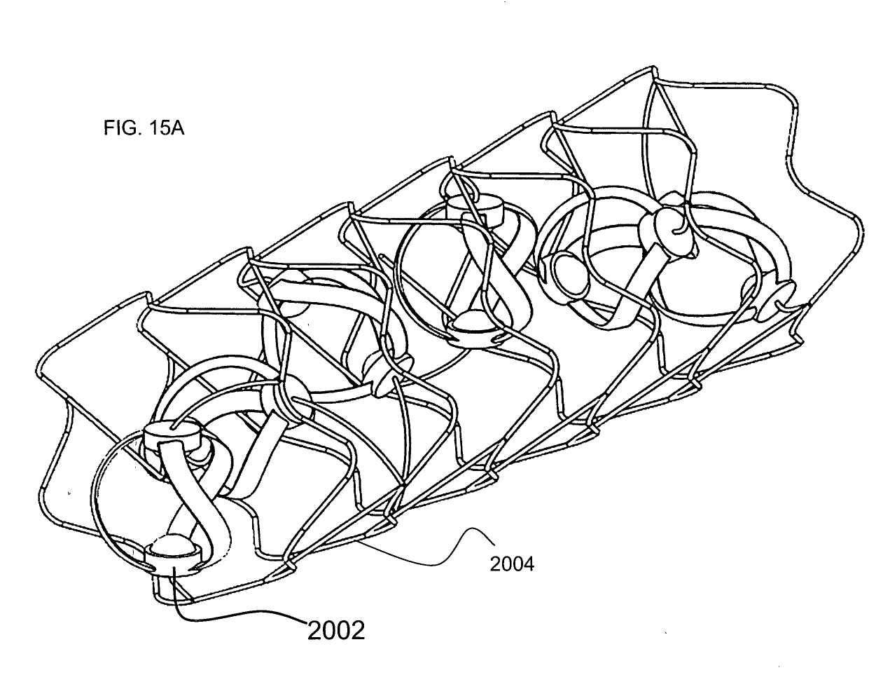

FIG. 14A depicts an exemplary device 1900 according to the present invention comprising vessel support structure 1902 and a plurality of rotating portions 1904; rotating portions 1904 are preferably placed in a stepwise and sequential manner within vessel support structure 1902. Optionally the plurality of rotating portions 1904 spans the inner diameter of vessel support structure 1902. Preferably the use of a vessel support structure 1900 provides an open corridor for blood flow while limiting the level of restenosis to a predefined level. Therefore, even in a situation where vessel support structure 1902 is experiencing restenosis a passageway or corridor is preferably maintained through which fluids may flow.

FIG. 14B depicts an exemplary embodiment of a vessel support structure 1912 preferably comprising at least one rotating portion 1914 and at least one filter portion 1916. Optionally rotating portion 1904 may be placed in bifurcation junction outside the lumen of vessel support structure 1912 along its outer surface of junction.

FIG. 14C depicts an exemplary embodiment of a vessel support structure 1922, preferably comprising at least one rotating portion 1924, at least one filter portion 1926. Optionally rotating portion 1924 may be placed along the plane of vessel support structure 1922 integrated within the vessel support structure.

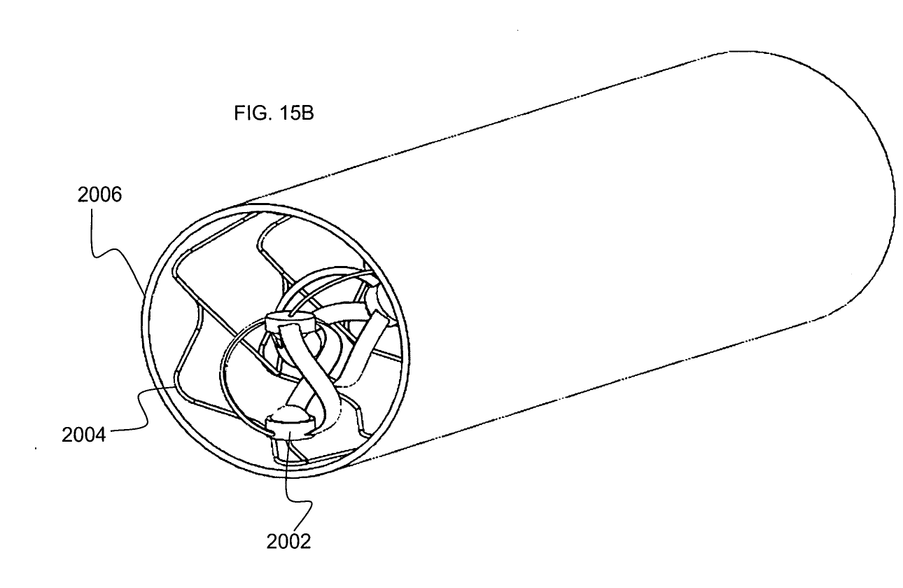

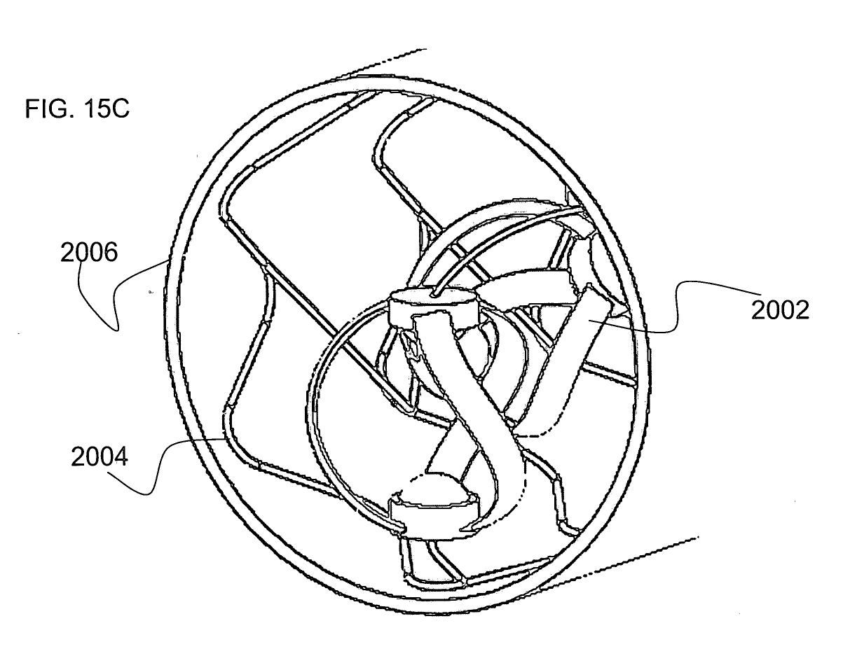

FIG. 15A provides a more detailed depiction of FIG. 14A, showing an optional embodiment according to the present invention wherein a plurality of rotating portions 2002 are preferably placed within a vessel support structure 2004, optionally and preferably providing full coverage of the support structure's lumen. FIG. 15B is a depiction of FIG. 15A as implemented within vessel 2006. Similarly, FIG. 15C, provides a perspective view of FIG. 15B.

While the invention has been described with respect to a limited number of embodiments, it will be appreciated that many variations, modifications and other applications of the invention may be made.

What is claimed is:

1. An implantable device for implantation to a vessel, comprising a vessel support structure for securing the device within said vessel, at least one rotating portion supported by said vessel support structure and an electronic portion for generating energy from said rotating portion.

2. The device of claim 1 further comprising windings for generating electrical current in the presence of a magnetic field.

3. The device of claim 1, wherein a flowing bodily fluid flows through said vessel causing said rotating portion to rotate and to generate energy.

4. The device of claim 3 wherein said rotating portion produces mechanical energy.

5. The device of claim 4 wherein said electronic portion converts mechanical energy to another form of energy selected from the group consisting of electrical, magnetic, optical, heat, electrostatic, UV, IR and RF.

6. The device of claim 5 wherein said electronic portion comprises an AC/DC or AC/AC power generator.

7. The device of claim 6, wherein said electronic portion further comprises an electrical power regulator.

8. The device of claim 6, wherein said electronic portion further comprises an electrical power filter.

9. The device of claim 6, wherein said electronic portion further comprises a power isolator.

10. The device of claim 1, wherein said electronic portion further comprises a communication interface.

11. The device of claim 1, further comprising a docking cradle.

12. The device of claim 1 wherein a medical device is coupled to and is at least partially powered by said electrical portion.

13. The device of claim 3 wherein one or more properties of said rotation portion are customized according to the implantation site or the properties of the flowing bodily fluid.

14. The device of claim 1 further comprising an energy converter coupled to said electrical portion for transmitting energy.

15. The device of claim 14, wherein said energy converter is wireless.

16. The device of claim 14, wherein said energy converter is wired.

17. The device of claim 1 further comprising an angioplasty balloon for disabling the rotating portion through inflation and spreading the rotating portion components against the vessel walls.

18. The device of claim 1 wherein said rotating portion is removable from the vessel support structure.

19. The device of claim 18 wherein said rotating portion is adapted to be removable with a catheter.

20. The device of claim 1 wherein said electrical portion is removable from the vessel support structure.

21. The device of claim 20 wherein said rotating portion is adapted to be removable with a catheter.

22. The device of claim 1 wherein said rotation portion is securely coupled to said vessel support structure using a lock assembly.

23. The device of claim 22 wherein said lock assembly is a pin lock or a turn lock assembly.

24. The device of claim 1 wherein said electrical portion is securely coupled to said vessel support structure using a lock assembly.

25. The device of claim 24 wherein recovery or retrieval is accomplished by disengaging a pin lock or turn lock assembly.

26. The device of claim 1 further comprising a sensor.

27. The device of claim 26 wherein said sensor is selected from the group consisting of a flow rate sensor, temperature sensor, blood sugar sensor, blood pressure, pH sensor, resistance sensor, insulin sensor and a piezoelectric sensor.

28. The device of claim 10 wherein said communication interface communicates to at least one external device selected from the group consisting of a modem, mobile phone, a display device, call center and external data storage.

29. The device of claim 10 wherein said communication interface communicates to an external medical device.

30. The device of claim 29, wherein said communication interface communicates to said external medical device wirelessly.

31. The device of claim 29, wherein said communication interface communicates to said external medical device through at least one wire.

32. The device of claim 29 wherein said communication interface transmits energy to an external medical device wirelessly.

33. The device of claim 11 wherein said docking cradle is physically connected to and transmits energy to an associated medical device.

34. The device of claim 5 wherein said optical energy is produced by an LED, or laser diode.

35. The device of claim 34 wherein said optical energy comprises a wavelength group comprising light selected from group of about 720-904 nm or other usable IR and UV wavelength.

36. The device of claim 35 wherein said optical energy is a laser diode producing laser energy comprising one or more of Argon, HeNe, GaAlAs, Nd: YAG.

37. The device of claim 1 wherein said rotation portion comprises a plurality of blades.

38. The device of claim 37 wherein said plurality of blades are in a concentric arrangement.

39. The device of claim 37 wherein at least one blade has variable width along the blade.

40. The device of claim 37 wherein said blades are helical or skewback airfoil shaped.

41. The device of claim 40, wherein said blades are warped into a spherical or elliptic shape.

42. The device of claim 37 wherein said plurality of blades has an open configuration.

43. The device of claim 37 wherein said plurality of blades has a closed configuration.

44. The device of claim 37 wherein said plurality of blades further comprise a material having magnetic properties.

45. The device of claim 37 wherein said material comprises magnetic material selected from the group consisting of permanent magnet, magnetic coating or material having magnetic properties.

46. The device of claim 1 wherein said vessel support structure comprises an anchor.

47. The device of claim 46, wherein said vessel support structure comprises a plurality of anchors.

48. The device of claim 46, wherein said anchor is flexible.

49. The device of claim 47, wherein said anchors are flexible.

50. The device of claim 49, wherein said anchors are connected with a shaft.

51. The device of claim 50 wherein the shaft is flexible.

52. The device of claim 37 wherein said at least one rotating portion further comprises a filter portion.

53. The device of claim 52, wherein said filter is within a blade rotation volume of said rotation portion.

54. The device of claim 53, wherein said filter is within a blade rotation volume wherein the blades are in an open configuration

55. The device of claim 53, wherein said filter is within a blade rotation volume wherein the blades are in a closed configuration.

56. The device of claim 1 wherein the vessel support structure is selected from the group consisting of a stent, a wire frame, a stent graft, ring structure, tripod structure and three winged structure.

57. The device of claim 1 in a collapsible linear form wherein the device components are folded onto themselves to allow delivery through a minimally invasive delivery system.

58. The device of claim 57 wherein the delivery system comprises a catheter, sheath and guidewire.

59. The device of claim 1 wherein one or more components comprise a material selected from the group consisting of 300 series stainless steels, platinum, platinum-iridium alloys, cobalt-chromium alloys, MP35N, unalloyed titanium, shape memory alloy (SMA), nickel titanium alloy and nitinol.

60. The device of claim 1 wherein one or more components comprise a material having a transition temperature around body temperature.

61. The device of claim 1 wherein one or more components comprise a shape memory polymer.

62. The device of claim 1 wherein one or more components comprise a material having a sensitivity or transition state governed by a change in at least one parameter selected from the group consisting of temperature, pH, electrical field, magnetic field.

63. The device of claim 1 wherein one or more components comprise a biodegradable material.

64. The device of claim 63 wherein the biodegradable material is selected from the group consisting of polylactic acid, polyglycolic acid (PGA), collagen, other connective proteins, magnesium alloys, polycaprolactone, hylauric acid, adhesive proteins, biodegradable polymers, biodegradable glass and bioactive glass.

65. A method of determining one or more cardiovascular parameters, comprising implanting an implantable device in a blood vessel, said implantable device comprising a rotating portion for rotating according to blood flow in said blood vessel, and measuring said rotating, wherein the one or more cardiac parameters are determined according to said rotating of said rotating portion.

66. The method of claim 65, wherein said one or more cardiovascular parameters are selected from the group consisting of blood flow rate, cardiac output, vessel occlusion, blood pressure, blood parameters.

67. The method of claim 65, wherein said implantable device further comprises a sensor for measuring said rotating.

68. The method of claim 67, wherein said sensor is an internal or external sensor.

69. The method of claim 67 wherein said sensor is an imaging device selected from the group comprising MRI or ultrasound.

70. The method of claim 65 wherein said implantable device further comprises an implanted medical device for being activated and operated according to a measurement from said sensor.

71. The device of claim 1 further comprising a dispenser for dispensing a medicament.

72. The device of claim 71, wherein said medicament is selected from the group consisting of heparin, heparin fragments, aspirin, coumadin, tissue plasminogen activator (TPA), urokinase, hirudin, streptokinase, antiproliferatives, methotrexate, cisplatin, fluorouracil, Adriamycin, antioxidants, ascorbic acid, carotene, vitamin B, vitamin E, antimetabolites, thromboxane inhibitors, non-steroidal and steroidal anti-inflammatory drugs, Beta and Calcium channel blockers, genetic materials, DNA fragments, RNA fragments, complete expression genes, carbohydrates, proteins, antibodies, monoclonal antibodies, polyclonal antibodies, lymphokines, growth factors, prostaglandins, and leukotrienes.

73. The device of claim 1 wherein one or more components are coated with a medicament selected from the group consisting of antiproliferatives, methotrexate, cisplatin, fluorouracil, Adriamycin, antioxidants, ascorbic acid, carotene, vitamin B, vitamin E, antimetabolites, thromboxane inhibitors, non-steroidal and steroidal anti-inflammatory drugs, Beta and Calcium channel blockers, genetic materials, DNA fragments, RNA fragments, complete expression genes, carbohydrates, proteins, antibodies, monoclonal antibodies, polyclonal antibodies, lymphokines, growth factors, prostaglandins, and leukotrienes.

74. The device of claim 6, wherein said electronic portion further comprises an electrical load.

75. The device of claim 6, wherein said electronic portion further comprises an energy converter.

76. The device of claim 1 further comprising a transmitter coupled to said electrical portion for transmitting telemetry data.

77. The device of claim 14, wherein said transmitter is wireless.

78. The device of claim 14, wherein said transmitter is wired.

79. The device of claim 1 wherein said rotation portion is securely coupled to said vessel support structure using a triggering mechanism applied by external source.

80. The device of claim 22 wherein said triggering mechanism is related to changes in the heat, cold, pH, electric or magnetic fields.

81. The device of claim 1 wherein said electrical portion is securely coupled to said vessel support structure using a triggering mechanism applied by external source.

82. The device of claim 1 wherein retrieval or recovery is accomplished by providing the triggering stimulus.

83. The device of claim 57 wherein once delivered the device is self expanding.

84. The device of claim 1 comprising charged materials.

85. The device of claim 84 wherein charged material are chosen from the group consisting of a polymer, piezoelectric material, or metals.

86. The device of claim 37 wherein the rotating device's spin is induced by an external source.

87. The device of claim 86 wherein said external source is selected from the group consisting of external electrical field, defibrillator, and external magnetic field.

88. The device of claim 1, wherein an internal surface of the vessel support structure is shaped as an airfoil for increasing blood flow through the rotating portion.
